(19) United States
(12) Patent Application Publication
(10) Pub. No.: US 2024/0079088 A1
(43) Pub. Date: Mar. 7, 2024

(54) METHODS AND COMPOSITIONS FOR GENOTYPING AND PHENOTYPING CANNABIS

(71) Applicant: LeafWorks Inc., Sebastopol, CA (US)

(72) Inventors: Kerin Bentley Law, Sebastopol, CA (US); Eleanor Johanna Kuntz, Penngrove, CA (US); Jugpreet Singh, Athens, GA (US); Laura Lee Klein, Sebastopol, CA (US); Nicholas Lee Batora, Dimondale, MI (US); Rishi Rajen Masalia, Phoenix, AZ (US)

(21) Appl. No.: 18/241,786

(22) Filed: Sep. 1, 2023

Related U.S. Application Data
(60) Provisional application No. 63/374,535, filed on Sep. 3, 2022.

(51) Int. Cl. G16B 20/20; G01N 33/00; G06V 10/143; G06V 10/40; G06V 10/764

(52) U.S. Cl. CPC G16B 20/20; G01N 33/0098; G06V 10/143; G06V 10/40; G06V 10/764

(57) ABSTRACT

Described herein are methods for identifying plant genomic regions that are optimized for cultivar screening, identifying an unknown Cannabis cultivar, verifying an identity of an unknown Cannabis cultivar, identifying genetic attributes of a Cannabis cultivar, and phenotyping a Cannabis cultivar. Such methods may be used to improve or alter cultivation practices, improve breeding efforts, determine the identity of source material, determine ancestry, estimate cultivar properties, and the like.

### CROSS REFERENCE TO RELATED APPLICATIONS

This application claims the benefit of priority under 35 U.S.C. 119(e) to U.S. Application No. 63/374,535 filed Sep. 3, 2022.

### TECHNICAL FIELD

This disclosure generally relates to methods and compositions for genotyping and phenotyping Cannabis, including hemp.

### BACKGROUND

Cannabis is a highly valuable economic crop for cannabinoid, fiber, and oil production. There are a wide variety of Cannabis cultivars with different traits and capacity to produce the chemical compounds and attributes for medical and industrial use. Notably, Cannabis is a cross-pollinated plant and has high genetic diversity, resulting in unstable traits throughout generations and further exacerbating the problem of trait and cultivar characterization. There is no reliable way to systematically characterize and compare cultivar identity and the traits of interest in cultivars. The identification and certification of cultivar identity and quality is missing, particularly when identifying plants requires lengthy and costly procedures for planting, growing, and visually and/or chemically characterizing cultivars of interest for favorable plant traits.

Modern agriculture has leveraged the power of sequencing tools to characterize and predict the properties of Cannabis cultivars. However, the methods only focus on measuring a few biomarkers instead of capturing a cultivar's overall uniqueness with respect to the entire Cannabis genome, and the genetic and phenotypic diversity in this genus. An integrative approach, combining genotyping supported by a large and diverse species genome database and phenotyping using imaging analysis, is lacking to characterize any Cannabis accessions in the modern Cannabis agriculture.

### SUMMARY

In one aspect, methods of identifying a Cannabis cultivar are provided. Typically, such methods include the steps of obtaining phenotypic data from one or more plants or plant parts from the cultivar; and/or obtaining genotypic data from one or more plants or plant parts from the cultivar; and assigning a cultivar designation based on the phenotypic data and/or the genotypic data, thereby identifying the cultivar.

In some embodiments, the phenotypic data is obtained by a requester (e.g., in the field). In some embodiments, the phenotypic data is obtained in a lab/remotely (e.g., via the grower transmitting a plant sample). In some embodiments, the phenotypic data is in a digital form (obtained via, e.g., 2D and/or 3D images or video) of the plant or a portion thereof. In some embodiments, the phenotypic data is compiled manually (via, e.g., a comprehensive checklist of character traits).

In some embodiments, the methods further include entering the phenotypic data into a phenotypic database. In some embodiments, the methods further include analyzing and, optionally, annotating, the phenotypic data.

In some embodiments, the phenotypic data comprises leaf size (e.g., length, width, etc.); plant size (e.g., canopy height and width, etc.); flower (e.g., color, size, shape, THC/CBD content, oil content, etc.); growth profile (e.g., days to maturity, days to flower, etc.); fiber density, tensile strength, biofuel efficiency, phytoremediation use, nutritive potential, nutrient content, ionomics, etc., etc.

In some embodiments, the genotypic data is obtained using polymerase chain reaction (PCR) (e.g., qPCR, dPCR, ddPCR), next generation sequencing (NGS) (e.g., genotype by sequencing (GBS), restriction site associated DNA sequencing (RADseq), long read sequencing, nanopore long read sequencing, Sanger sequencing), restriction fragment length polymorphism (RFLP) analysis, oligonucleotide probes SNP chip array, microarray, and combinations thereof. In some embodiments, the genotypic data comprises genetic analysis (e.g., SNPs), transcriptional analysis, translational analysis, copy number variation analysis metabolomics analysis, proteomic analysis, epigenetic analysis, or combinations thereof. In some embodiments, the methods further include entering the genotypic data into a genotypic database. In some embodiments, the methods further include analyzing and, optionally, annotating, the genotypic data. In some embodiments, the methods further include determining genetic relationship information from the genotypic data. In some embodiments, the genotypic data is used to determine genetic relationship information of the cultivar. In some embodiments, the genotypic data is used to determine features of the genetic makeup of the cultivar and/or an evolutionary relationship of the cultivar with other taxa.

In some embodiments, the methods further include entering the assigned cultivar designation into a database. In some embodiments, the methods further include transmitting the assigned cultivar designation to a requester or recipient. In some embodiments, the requester or recipient is a grower, a government/regulatory agency, a dispensary, an individual, law enforcement, a researcher, a company, a breeder, etc. In some embodiments, the assigned cultivar designation comprises one or more designations selected from species, subspecies, varieties, subvarieties, forma, and subforma.

In some embodiments, the methods further include obtaining breeding and/or ancestry information. In some embodiments, the breeding and/or ancestry information is obtained from label information, historical information, plant trait data, plant genetic information, and combinations thereof. In some embodiments, the methods are performed in duplicate or triplicate. In some embodiments, the methods are at least partially automated. In some embodiments, the methods use a processor.

In some embodiments, the methods further include providing, characterizing, confirming or denying breeding information. In some embodiments, the methods further include providing, characterizing, confirming or denying ancestry information. In some embodiments, the methods further include providing, characterizing, confirming or denying cultivar identity information. In some embodiments, the methods further include providing, characterizing, confirming or denying supply chain information. In some embodiments, the methods further include verifying/certifying the information.

In another aspect, methods of identifying a Cannabis plant or portion thereof are provided. Such methods typically include the steps of obtaining genotypic data from the plant or portion thereof; and comparing the genotypic data obtained from the plant or portion thereof to reference genotypic data for Cannabis spp., thereby identifying the Cannabis plant or portion thereof.

In some embodiments, the genotypic data is obtained by sequencing genomic DNA from the plant or portion thereof. In some embodiments, the genotypic data is obtained by RAPD, AFLPs, RFLPs, or combinations thereof. In some embodiments, the genotypic data is obtained by reduced representation sequencing, whole genome sequencing, exon sequencing, short or long read sequencing, transcriptome sequencing, epigenetic information, or combinations thereof.

In some embodiments, the methods further include validating or certifying the identity of the Cannabis plant or portion thereof. In some embodiments, the methods further include determining if the Cannabis plant is clonal, a sibling, or a distant relative with respect to a reference plant or reference plant material.

In still another aspect, methods of identifying a Cannabis plant are provided. Such methods typically include the steps of obtaining genotypic data from the plant; and comparing the genotypic data from the plant to one or more databases of genotypic data, thereby identifying the Cannabis plant.

In some embodiments, the genotypic data is obtained by sequencing genomic DNA from the plant or portion thereof. In some embodiments, the genotypic data is obtained by reduced representation sequencing, whole genome sequencing, exon sequencing, short or long read sequencing, or combinations thereof.

In some embodiments, the genotypic data is used to evaluate heterozygosity, genetic distance, and/or uniqueness.

In some embodiments, the identifying comprises identification of most likely cultivar, identification of most closely related cultivar with genetic similarities of certain features or attributes, identification of least closely related cultivar with genetic similarities of certain features or attributes. In some embodiments, the identifying comprises identification of relevant phenotypic traits. In some embodiments, the methods further include reporting relevant genotypic and/or phenotypic traits.

In yet another aspect, methods of identifying or characterizing a Cannabis plant are provided. Such methods typically include the steps of obtaining at least one image of the Cannabis plant; determining a criteria for at least one phenotypic trait using the at least one image of the Cannabis plant; and comparing the criteria for the at least one phenotypic trait of the Cannabis plant with at least one database of phenotypic traits, thereby identifying or characterizing the Cannabis plant.

In some embodiments, the images are of whole plants. In some embodiments, the images are of plant tissues. In some embodiments, the images are digital images. In some embodiments, the images are obtained at a plurality of wavelengths.

In some embodiments, the database of phenotypic traits comprises Cannabis images. In some embodiments, the at least one database of phenotypic traits comprises images of herbarium specimens. In some embodiments, the phenotypic traits comprise the size, shape and color of the overall plant, leaf, seed, and stem. In some embodiments, the phenotypic traits comprise leaf size (e.g., length, width, etc.); plant size (e.g., canopy height and width, etc.); flower (e.g., color, size, shape, THC/CBD content, oil content, etc.); growth profile (e.g., days to maturity, days to flower, etc.); etc.

In some embodiments, the comparing is across a plurality of phenotypic traits. In some embodiments, the method is at least partially automated.

One aspect of the present disclosure is directed to a method of identifying a set of genomic regions that are optimized for cultivar screening. In some embodiments, the method comprises: identifying a plurality of genomic regions based on a target genome; aligning the plurality of genomic regions from a cultivar to the target genome; extracting a first subset of genomic regions from the plurality of genomic regions based on the aligning; integrating the first subset of genomic regions to a plurality of plant genomes in a database; determining a read depth of each plant genomic region of the plurality of plant genomes in the database that represented at least one of the first subset of genomic regions; and extracting a second subset of genomics regions from the first subset of genomic regions when the read depth of each aligned plant genomic region is equal to or greater than a predefined threshold.

In any of the preceding embodiments, the first subset of genomic regions each span from about 100 bp to about 150 bp.

In any of the preceding embodiments, the predefined threshold is greater than equal to about 5 reads.

In any of the preceding embodiments, the target genome is CBDRX genome.

In any of the preceding embodiments, at least one genomic region of the first subset of genomic regions is comprised of two overlapping genomic regions of the plurality of the genomic regions.

In any of the preceding embodiments, the method further comprises determining a diversity of the second subset of genomic regions.

In any of the preceding embodiments, the diversity comprises an indication of at least one SNP in each of the second subset of genomic regions.

In any of the preceding embodiments, the diversity comprises determining a distribution across a plurality of chromosomes of each of the second subset of genomic regions.

In any of the preceding embodiments, the method further comprises stratifying the second subset of genomic regions based on the read depth of the aligned plant genomic regions.

Another aspect of the present disclosure is directed to a method of verifying an identity of the Cannabis cultivar. In some embodiments, the method comprises: genetically typing an unknown Cannabis plant sample; generating a genetic pattern specific to the unknown Cannabis plant sample, based on a predefined set of genomic regions; comparing the genetic pattern specific of the unknown Cannabis plant sample to a reference Cannabis plant genetic pattern; and outputting an indication of relatedness between the reference Cannabis plant genetic pattern and the genetic pattern specific to the unknown Cannabis plant sample.

In any of the preceding embodiments, the genetic typing comprises Restriction site Associated DNA sequencing.

In any of the preceding embodiments, the genetic typing comprises double digest Restriction site Associated DNA sequencing.

In any of the preceding embodiments, the genetic typing comprises double Restriction site Associated DNA sequencing or triple Restriction site Associated DNA sequencing.

In any of the preceding embodiments, the predefined set of genomic regions were identified by: identifying a plurality of genomic regions based on a target genome; sequencing a plurality of genomic regions based on a predefined set of genomic regions from a target cultivar genome; aligning the plurality of genomic regions to the target genome; extracting a first subset of genomic regions from the plurality of genomic regions based on the aligning; integrating the first subset of genomic regions to a plurality of plant genomes in a database; determining a read depth of each plant genomic region of the plurality of plant genomes in the database that aligned with at least one of the first subset of genomic regions; and extracting a second subset of genomics regions from the first subset of genomic regions when the read depth of each aligned plant genomic region is equal to or greater than a predefined threshold.

Another aspect of the present disclosure is directed to a method of identifying a Cannabis cultivar. In some embodiments, the method comprises: genetically typing an unknown Cannabis plant sample; generating a genetic pattern specific to the unknown Cannabis plant sample, based on a predefined set of genomic regions; comparing the genetic pattern specific to a database of known Cannabis plant genetic patterns; and outputting an identity or one or more attributes of the unknown Cannabis plant sample based on the comparison.

In any of the preceding embodiments, the genetic typing comprises Restriction site Associated DNA sequencing.

In any of the preceding embodiments, the genetic typing comprises double digest Restriction site Associated DNA sequencing.

In any of the preceding embodiments, the genetic typing comprises double Restriction site Associated DNA sequencing or triple Restriction site Associated DNA sequencing.

In any of the preceding embodiments, the predefined set of genomic regions were identified by: identifying a plurality of genomic regions based on a target genome; aligning the plurality of genomic regions from a cultivar to the target genome; extracting a first subset of genomic regions from the plurality of genomic regions based on the aligning; aligning a plurality of plant genomes in a database to the first subset of genomic regions; determining a read depth of each plant genomic region of the plurality of plant genomes that aligned with at least one of the first subset of genomic regions; and extracting a second subset of genomic regions from the first subset of genomic regions when the read depth of each aligned plant genomic region is equal to or greater than a predefined threshold.

Another aspect of the present disclosure is directed to a computer-implemented method of phenotyping a Cannabis cultivar. In some embodiments, the computer-implemented method is performed by a processor and comprises: receiving an input image of the Cannabis cultivar; identifying a plurality of regions of interest in the input image; identifying one or more traits in one or more of the plurality of regions of interest; comparing the one or more traits to a database of known Cannabis cultivars, wherein the database is configured to link each trait to a property of the Cannabis cultivar; and outputting an indication of one or both of the property and the one or more traits.

In any of the preceding embodiments, identifying the plurality of regions of interest comprises identifying one or more physical landmarks in an x-coordinate frame and a y-coordinate frame.

In any of the preceding embodiments, the one or more traits comprise a number of leaflets per leaf, a branching structure, a canopy structure, a leaf shape, a leaf color, a presence of powdery mildew detection, or a combination thereof.

In any of the preceding embodiments, the one or more traits are linked to the property that is selected from the group consisting of: a plant spacing parameter, an airflow parameter, a light penetration parameter, a yield parameter, or a combination thereof.

In any of the preceding embodiments, the one or more traits comprise a leaf color, a presence of powdery mildew detection, or a combination thereof.

In any of the preceding embodiments, the one or more traits are linked to the property that is selected from the group consisting of: a reflectance parameter, a light penetration parameter, an inflorescence quantification parameter, a bud quantification parameter, a trichome parameter, a leaf quantity, a yield parameter, or a combination thereof.

In any of the preceding embodiments, the one or more traits comprise at least a leaf shape.

In any of the preceding embodiments, the one or more traits are linked to the property that is selected from the group consisting of: a plant spacing parameter, a plant size parameter, a light penetration parameter, a biomass parameter, a yield parameter, or a combination thereof.

In any of the preceding embodiments, the indication comprises a trait stability indication.

In any of the preceding embodiments, the indication comprises weighting the property as environmentally controlled.

In any of the preceding embodiments, the indication comprises weighting the property as genetically controlled.

There are numerous advantages to the methods described herein. For example:
The cultivar registration methods described herein can be used in the enforcement of Material Transfer Agreement (MTA).
The cultivar registration methods described herein can be used in Appellation applications to show that a terroir produces a higher quality product (e.g., performing registration of the cultivar in one environment vs another can demonstrate how phenotypes can change even when genotypes remain the same).
The cultivar registration methods described herein can be used as a form of timestamp tied to a physical plant to demonstrate possession of a specific cultivar or plant.
The cultivar registration methods described herein can be used to establish a cultivar as certified reference material (e.g., a gold standard), which can be used for validating label claim accuracy and transparency.
The cultivar registration methods described herein can be used for auditing and enforcement (e.g., genetic tracking and tracing of plants or plant material).
The cultivar registration methods described herein can be combined with Artificial Intelligence to determine other unique phenotypic or genotypic features of a plant.
Some groups perform only genotyping in Cannabis to establish that someone has possession of a cultivar, however an individual can submit flower from a dispensary under their name, but that does not mean the individual has any ownership claim to the plant. This is flawed. As a better alternative, the cultivar registration methods described herein can be used to connect genotypes to the physical plant, and do a phenotypic assessment.
Everyone in the Cannabis industry wants to be able to identify or distinguish cultivars, but focusing solely on genotype or, alternatively, solely on phenotype, does little to characterize and distinguish a cultivar that is similar to other cultivars.
The advantages of the supply chain certification and the double check test are being able to confirm the identity of a plant or plant part during shipment, at receipt, ensure cultivar labels remain accurate, and throughout the cultivation and processing workflow while making sure nothing has adulterated the product.
The supply chain certification methods can be used to enforce contracts and detect the improper sharing of clones, seeds, and/or plant cuttings.
The database used in the cultivar ID testing methods is extensive, including commercial varieties and also landraces, making the cultivar comparisons described herein (e.g., most related, least related) meaningful and more accurate. This database contains DNA samples from certified reference material held in an herbarium (Canndor Herbarium) that can be referenced back to a specific cultivar or strain.
The advantages of the 2D imaging test are its novelty in Cannabis. There is not a platform that uses phenotypic data for Cannabis in the form of an image scan.
The 2D imaging test also can be expanded into any number of additional traits, can incorporate machine learning to “learn” during the scanning, and can use 3D imaging including other visual formats such as hyperspectral.

As used in the description and claims, the singular form “a”, “an” and “the” include both singular and plural references unless the context clearly dictates otherwise. For example, the term “trait” or “genomic region” may include, and is contemplated to include a plurality of traits or a plurality of genomic regions or genetic markers covered by the plurality of genomic regions. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety, as if each individual publication or patent application was specifically and individually indicated to be incorporated by reference in its entirety.

### DESCRIPTION OF DRAWINGS

FIG. 1 illustrates one embodiment of a system for genotyping and/or phenotyping a plant.

FIG. 2 illustrates one embodiment of a method for identifying a plurality of genomic regions (e.g., markers) that can be used to genotype a plant.

FIG. 3 illustrates one embodiment of a genotypic method for determining the identity of an unknown plant. Embodiments described herein include supply chain certification or double check test.

FIG. 4 illustrates one embodiment of a genotypic method for identifying a plant (e.g., identifying the cultivar of the plant).

FIGS. 5A-5C each illustrate an embodiment of a phenotypic method for identifying a Cannabis cultivar.

FIG. 6 illustrates one embodiment of a method of phenotypically identifying a Cannabis cultivar.

FIG. 7 is a flow chart of the steps performed by the software, including inputs and outputs.

The illustrated embodiments are merely examples and are not intended to limit the disclosure. The schematics are drawn to illustrate features and concepts and are not necessarily drawn to scale. Like reference symbols in the various drawings indicate like elements.

### DETAILED DESCRIPTION

Disclosed herein are systems and methods for genotypic and/or phenotypic analysis of Cannabis cultivars. For example, in some embodiments, genotypic and/or phenotypic analysis can be used to identify an unknown cultivar or plant or plant trait, to identify an unknown cultivar or plant relative to one or more known cultivars or plants, to verify an identity of a cultivar or plant relative to a known cultivar or plant (e.g., in supply chain management), or the like. As used herein, Cannabis refers to any species, subspecies, varieties, subvarieties, cultivars, forma, or subforma of the genus Cannabis, including any and all hemp cultivars.

Various plant parts can be used for analyses in the methods described herein. For example, various samples that can be used include, but are not limited to, an extrapetiolar sample (i.e., outside of, but close to, the petiole), a perianth sample (i.e., calyx and corolla of a flower, collectively), a petiole sample (i.e., leaf stalk), a pistillate sample (i.e., bearing pistils but not stamens), a female flower sample, a leaf punch, a plant sample on a chemically treated filter paper designed to degrade proteins such as Whatman™ paper, a staminate sample (i.e., bearing stamens but not pistils), a stipule sample (i.e., one of a pair of leaf-like appendages found at the base of the petiole in some leaves), a whole leaf sample, a partial leaf sample, a stem sample, a root sample, or combinations thereof.

In some embodiments, the methods described herein can include isolating genetic material (e.g., genomic DNA or specific regions of the genome) from a plant. Isolating genetic material can include, but is not limited to: homogenizing a plant sample (e.g., seed, leaf, stem, flower, etc.), creating a tissue lysate using, for example, a lysis buffer (e.g., an ionic detergent, cetyltrimethylammonium bromide (CTAB) buffer, sorbitol, TENT (Tris-EDTANaCl-TritonX100) buffer, or other suitable buffer or detergent), DNA extraction (e.g., using phenol:chloroform:isoamyl alcohol in, e.g., Qiagen® kits, Tris-EDTA buffer, high salt-CTAB buffer, or other extraction methods or buffers), and DNA precipitation (e.g., using sodium acetate, salt-based solution, isopropanol, ethanol, or similar). The plant sample may be homogenized under cryogenic conditions, on ice, or otherwise homogenized to preserve genetic material and minimize degradation. It would be appreciated that the entire process of isolating genetic material or one or more steps thereof can be automated.

In some embodiments, various metrics such as diversity, uniqueness, relatedness, matching, or the like can be used to describe or identify a plant or cultivar.

As used herein, “heterozygosity” refers to an estimate of the degree of genetic variation within a plant sample relative to a database or a plurality of plant samples. Heterozygosity is calculated by either (1) normalizing the count for heterozygous sites to all the SNPs detected (standardized to the number of sites that are included in the comparison minus the heterozygous minimum divided by the heterozygous range (difference between max and min)); or (2) calculating the number of heterozygous states per sample (cultivar of interest) across all or a subset of the genomic sites in the database and plotting this against all or a subset of the samples in the database. At least one problem identified by the inventors is that cannabis cultivars have highly heterozygous genomes, but there are no developed tools specifically for Cannabis. Highly heterozygous (e.g., usually cross-pollinated) plants do not produce consistent phenotype(s) over generations for traits of interest (e.g., yield, THC content, etc.). A technical solution for this technical problem, as described herein, is to detect where a Cannabis cultivar is heterozygous at specific genomic sites, so that less heterozygous plants may be selected for propagation, thus yielding more predictable phenotypes and a consistent resulting product in the subsequent generations. Further, a heterozygosity analysis may indicate the phenotypic and/or genetic stability of a plant or cultivar sample. For example, plant samples with low heterozygosity will be more phenotypically stable than the ones with high heterozygosity in the subsequent generations.

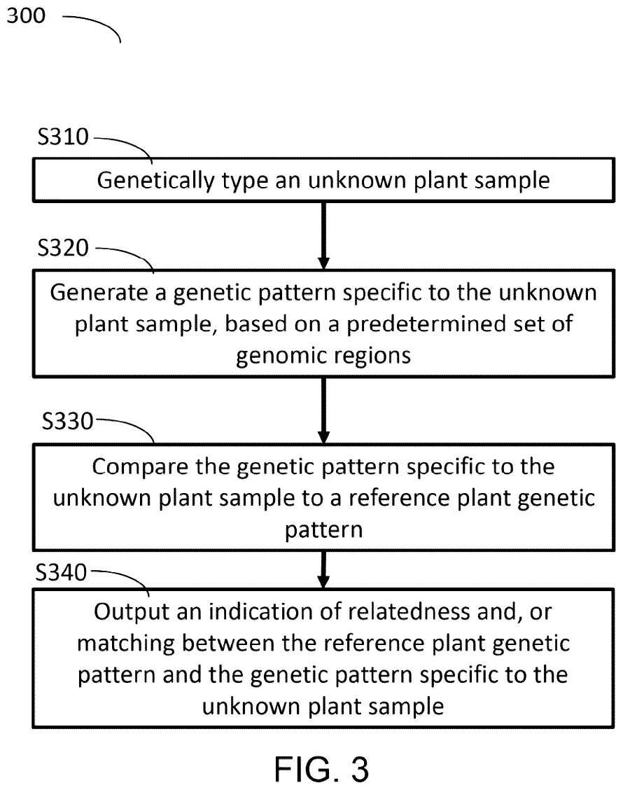

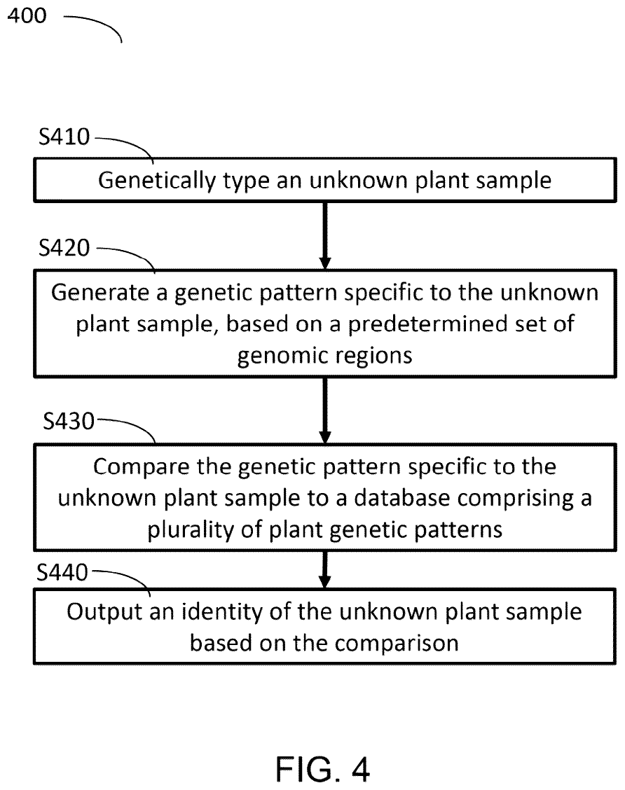

As used herein, “uniqueness” refers to how rare or common a plant sample is relative to other cultivars, for example in a database. As used herein, “relatedness” refers to how genetically similar an unknown plant sample to all samples in the database. Uniqueness and relatedness refer to the metrics generated by calculating the Identity by State (MS) using a pair-wise comparison of SNPs between samples in a database to determine how similar two samples are based on sequence. The pairwise comparison further can be used to determine known or unknown clonal or familial relationships between samples (i.e., relatedness). Uniqueness or relatedness can be determined, for example, by performing a pairwise comparison at genomic regions or sites determined at a second subset of genomics regions (e.g., see FIG. 3, FIG. 4), between a cultivar of interest to the unique genetic patterns or genotyping for one or more, a plurality of, or all the cultivars in the database. For example, one can determine if and how much the genotype of a cultivar is different or not compared to the genotype of other cultivars at any specific locus in a database with varying scoring rules according to the genomic region features selected, as described elsewhere herein. Alternatively, uniqueness or relatedness or diversity may be determined by comparing each of the second subset of genomic regions (e.g., see FIG. 4) to the database, where any differences in each region are recorded as unique values. The values are then normalized by standardizing to the number of sites that are included in the comparison, minimum value in the database, and/or the range (difference between max and min) (e.g., hetuniq$Norm_Uniq=(hetuniq$UniqScore−uniqmin)/uniqrange). A lower score means more relatedness (when compared between two samples) or less uniqueness (when compared to the database).

A relatedness calculation can be used to determine whether the plant sample or cultivar sample has a clonal match, related match (e.g., half sibling, full sibling, parent, offspring, etc.), or no-match. For example, for verifying an identity of a cultivar using supply chain verification, the comparison may be between a plant or cultivar sample and a specific sample or group of samples in a database (e.g., Cannabis samples cross-checked to a Cannabis cultivar database or herb samples cross-checked to an herb species database). Further for example, for registering a potentially new cultivar, the comparison may be used to determine what a plant or cultivar sample is most similar to or most dissimilar from relative to a plurality of samples in the database. Further still for example, for cultivar identification, the comparison may be used to determine what a plant or cultivar sample is most similar to or most dissimilar from relative to a plurality of samples in a database.

As used herein, “diversity” refers to the nucleotide and/or genetic diversity in a population. Diversity is determined by the number of nucleotide differences and/or the size and/or the number of structural genomic differences between any DNA sequence pairs for all the individuals in a population and is represented by pi (it). Diversity for a sample is determined by comparing the DNA sequence of that sample to a set reference genome and calculated by measures such as pi, Watterson estimator (theta; Co), Tajima D's, Fst, etc. This measure may, additionally or alternatively, be plotted against a plurality of cultivars or samples in a database to determine a distribution of such measures (it, Co) across the database. Calculating diversity may include calculating a diversity at each region and then calculating an overall score for a cultivar or plant sample. Alternatively, calculating diversity may include calculating an aggregate score across all or a subset of regions across all or a subset of cultivars in a database. For example, diversity may refer to a degree of heterozygosity, a SNP number, a SNP distribution across genomes, structural variations including insertions or deletions, inversions, translocations, degree of genome recombination, number of variants at a genomic locus, polymorphism or rate of polymorphism at genetic or epigenetic markers, proportion of polymorphic loci, number of alleles and/or allelic richness, average number of alleles per locus, frequency of variant alleles, etc.

As described herein, the methods may be computer-implemented (e.g., a computer-readable medium having instructions stored thereon, the instructions being executed by a processor or one or more processors) or a mix of laboratory methods and computer-implemented methods. For example, genetic material may be isolated through various laboratory methods, and genetic region analysis and comparison may be performed through computer-implemented methods. Further for example, phenotypic typing may be a computer-implemented process or a mix of user input observations and computer-implemented methods. The processor may be a local processor (e.g., desktop, mobile computing device, workstation, etc.) or a remote processor (e.g., server) or a combination of both where more than one processor is used. For image analysis, the processor may be communicatively coupled to an image sensor (e.g., integrated into the same device and electrically connected or in separate devices such that information is communicated between devices via a coil, antenna or the like), such that the processor is configured to receive an input sensor signal, or the processor may access an image from memory, such that the processor is configured to receive an input image.

The processor(s) may include one or more hardware processors, including microcontrollers, digital signal processors, an application specific integrated circuit (ASIC), a field programmable gate array (FPGA) or other programmable logic device, discrete gate or transistor logic, discrete hardware components, or any combination thereof designed to perform the functions described herein and/or capable of executing instructions, such as instructions stored by the memory. The processor(s) may also be able to execute instructions for performing communications amongst databases, sensors (e.g., image sensor), data processing modules, mobile computing devices, and/or third-party integrations.

### Cultivar Registration

Cultivar registration refers to an industry report that combines phenotypic and genotypic information (e.g., data) to characterize and define a Cannabis cultivar as well as provide information on its genetic and phenotypic attributes and uniqueness. Cultivar registration allows for 1) creation of certified reference material; 2) Plant Variety Protection (PVP) Certificate application information, support and enforcement; 3) contract auditing and enforcement such as material transfer agreements; 4) establish a record of a cultivar as a baseline for breeding new varieties and prove the presence of a new cultivar (e.g., for PVPs); 5) create an indisputable record of possession in the market and ownership; 6) create historical record for preservation of biodiversity; and/or 7) create physical record proving differences in attributes among portfolio of plants in a company.

As part of the phenotyping process, a voucher can be generated based on physical attributes for, e.g., leaf shape, branching, color, and/or other physical characteristics. A voucher is a reference material and provides a standard of proof for plant identity. A voucher typically is a pressed, dried specimen of a plant that has been mounted on archival paper. A label identifies and describes the plant, including information about when and where the plant was collected, its habitat or cultivation method, phenotypic information such as color, chemical profiles or yield amounts, the name of the collector, original breeder, steward or farm. For industrial applications, the label also can include batch numbers, lot numbers, and be used as a reference to track, trace or audit plants as needed in the event of supply chain discrepancies.

In some embodiments, a voucher regarding physical attributes can be generated and/or provided by a third party (e.g., an herbarium (e.g., Canndor Herbarium)) while, in some embodiments, a voucher regarding physical attributes can be generated and/or provided as a part of the cultivar registration service. Vouchers regarding physical attributes can be used in combination with genotyping in the cultivar registration process, or vouchers regarding physical attributes can be used in combination with both genotyping and 2D imaging in the cultivar registration process. A voucher can be a standalone plant record that can later be used for genotyping and/or phenotyping, or a voucher can be an integral part of the phenotyping process.

Phenotypic properties can be obtained through digital imaging. 2D images can be used to extract trait values using a custom code created in PlantCV (built on open-source platform, OpenCV (general image analysis). Phenotypes are evaluated, quantified (if relevant), and can be compared to a database (e.g., created from 2D images of herbarium specimens), if desired. The phenotypic analysis can determine how rare or unique an attribute is. More information about the phenotypic properties is provided below.

Genotypic properties are usually determined by sequencing genomic DNA. Any number of methods can be used to sequence genomic DNA including, for example, whole genome sequencing, reduced representation sequencing, restriction site associated DNA sequencing, single restriction site associated DNA, double restriction site associated DNA sequencing, multiple restriction site associated DNA sequencing, amplicon sequencing, probe sequencing, targeted region sequencing (such as exosomes), or the like.

If necessary prior to sequencing, genomic DNA can be extracted from plant tissue prior to sequencing. DNA extraction methods are known in the art, and can include the use of one or more commercial kits and/or reagents (e.g., Qiagen, Axygen, Promega, BioRad). DNA analysis includes assigning metrics to the sample itself and evaluating how those metrics relate to the database of other samples within and across cultivars in the species to make inferences (e.g., confirm or deny) about breeding, ancestry, cultivar identity, or supply chain, to name but a few. As discussed herein, the genotypic analysis portion of cultivar registration can determine the most similar or dissimilar sample(s) in a large database of Cannabis sequences. Also as discussed herein, pairwise comparison can be used to determine the threshold for similar and dissimilar samples.

A genotypic report is created indicating the level of heterozygosity in the genome and providing information such as how the level of heterozygosity compares across cultivars in the species, the uniqueness of the sample across cultivars in the species, and the most closely related and least closely related samples from the database.

Additional information can be obtained about the cultivar, e.g., the pedigree history of the cultivar, how the cultivar is grown, e.g., for optimal performance, and traits that cannot be gleaned from the herbarium vouchers, by interviewing the individual or entity requesting the cultivar registration (e.g., a grower, a breeder, etc.). These additional traits, which include, without limitation, main stem diameter grooves (e.g., presence/absence), color (e.g., qualitative range), pubescence (e.g., qualitative description), hollowness (e.g., qualitative description), average length between internodes (e.g., branching points), canopy height and width, cola (e.g., largest inflorescence at the top of the plant) length and width, seed color and/or marbling, seed size, average seed weight, morphological description, medicinal uses, olfactory characteristics, chemistry profiles, processing categories (e.g., fresh flower, extraction, hash, etc.), disease resistance and/or susceptibility, and/or proportion of males, females, or hermaphrodites, can have applications in genetic mapping.

Chemistry profiles can be obtained for a cultivar using known methods. Knowing the chemical profile of a cultivar can allow growers/breeders to source material having specific characteristics. Chemical information can also be used in genetic mapping for identification and or validation of genes that predict chemical production output.

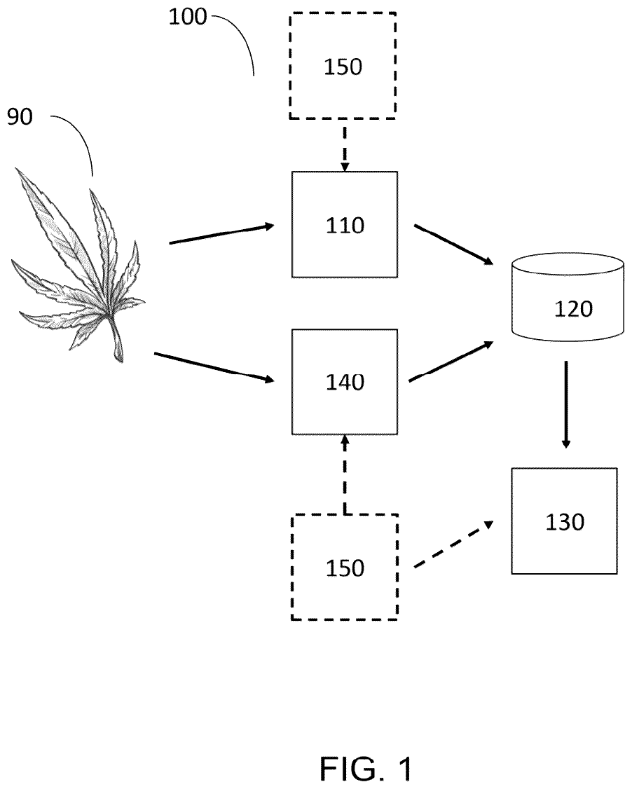

FIG. 1 shows an exemplary embodiment of a system for genetically and/or phenotypically typing a plant. In some embodiments, genetic material is isolated from a plant 90 or plant tissue at block 110. A plurality of genomic regions within the genetic material can be compared to a plurality of genomic regions within a database 120 or to genomic regions from one or more known samples, the details of such comparison is described herein. The output 130 of the comparison may comprise a report, a recommendation, an indication, one or more parameters that are configured to be displayed on a graphical user interface of an associated device (e.g., mobile computing device, remote device, workstation, laptop, etc.), or the like. Typically, the output includes an assignment of a cultivar designation based on the genotypic data and phenotypic data provided.

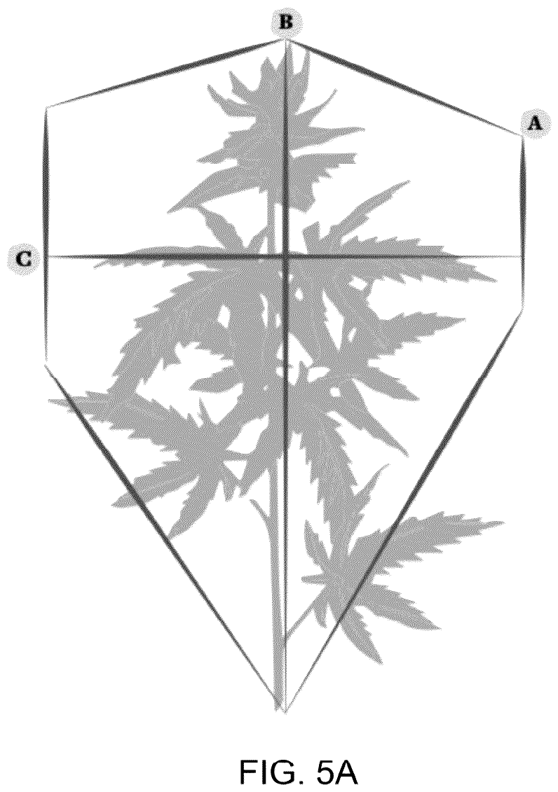

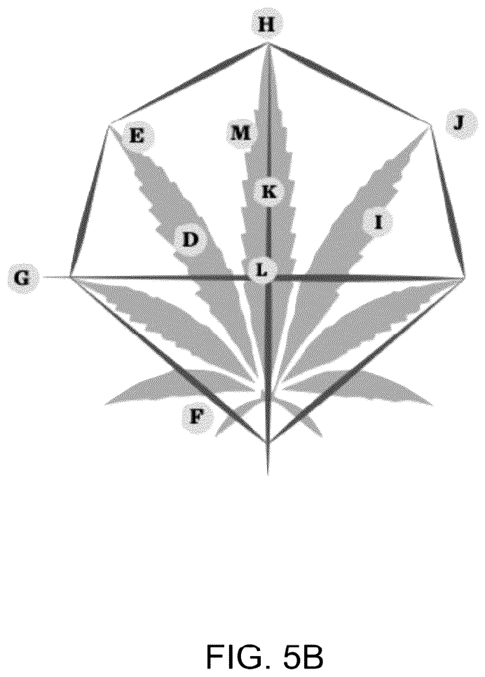

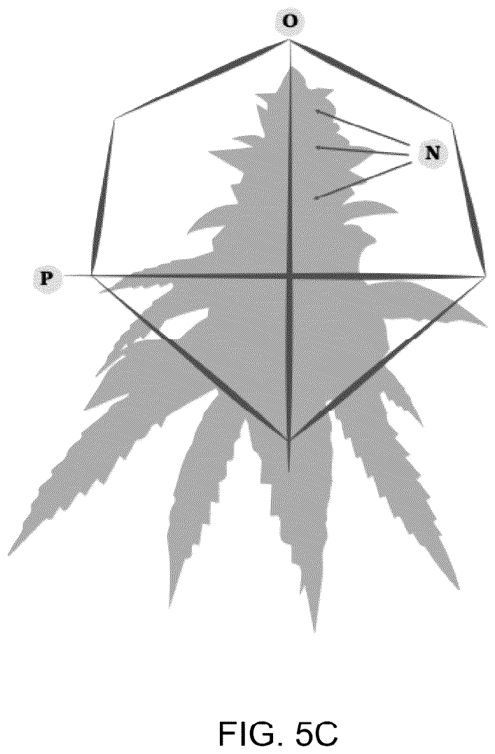

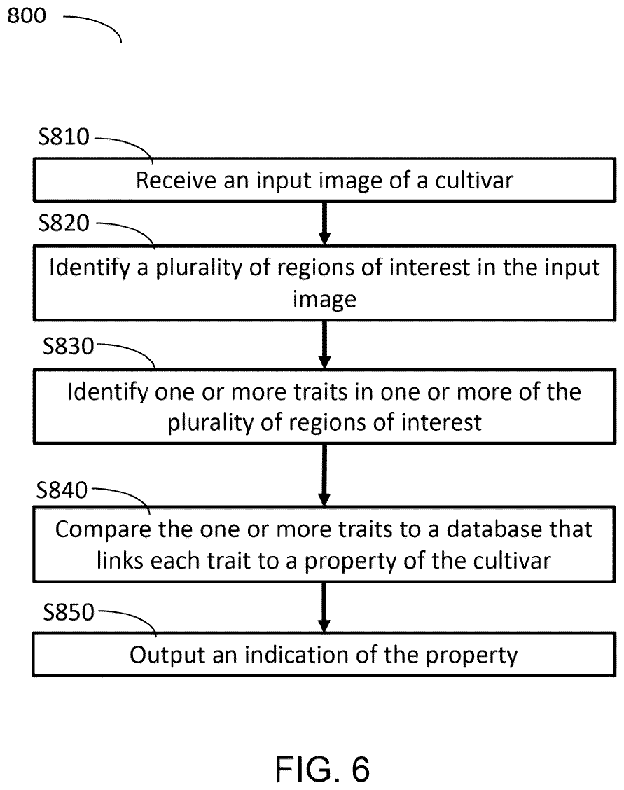

Additionally, or alternatively, the sequence of a plurality of genomic regions of interest within the plant 90 genome can be determined and input into a plant phenotyping pipeline 140, as described herein. Various traits and/or properties of the plant or cultivar may be compared to a plurality of traits and/or properties in a database 120 or to one or more or a plurality of traits and/or properties of a known cultivar, the results of which are described with respect to FIGS. 5A-6. The output 130 of the comparison of the traits or properties may include a report, a recommendation, an indication, one or more parameters that are configured to be displayed on a graphical user interface of an associated device (e.g., mobile computing device, remote device, workstation, laptop, etc.), or the like. Although traits and properties are described with respect to plant phenotype, it should be appreciated that genomic regions, for example their distribution, location, structure, etc., may also be considered a trait, such that these genomic regions may indicate, alone or in aggregate, various properties of the plant, some of which are outlined in Table 1 and elsewhere herein. In such embodiments, analyzing a second subset of genomic regions may include outputting an indication of a trait or a property of the cultivar or species for the purposes of understanding, identifying and predicting traits of interest.

Although database 120 in FIG. 1 is shown as one database, one of skill in the art will appreciate that database 120 may comprise more than one, one or more, two, or a plurality of databases. For example, a first database may comprise genomic sequencing data, and a second database may comprise trait and/or property data, as one non-limiting example.

In some embodiments, user input 150 is optionally received as input into the system at any one or more of blocks 110, 140, or 130 and used to further perform identification, analysis, or outputs related to a plant or cultivar. For example, user input may include, but is not limited to: chemical analysis data, plant ancestral data, growing habits, a botanical description, a grow location, mother plant name, father plant name, mother plant trait(s), father plant trait(s), grower history, plant history, general cultivation characteristic(s) (e.g., positive characteristics, challenging characteristics, etc.), cultivation variable(s) (e.g., outdoor cultivation, indoor cultivation, greenhouse cultivation, mixed light cultivation), pest or pathogen resistance or susceptibility, morphological description(s), phenotypic description(s), medicinal use(s), user experience(s), user profile(s), or the like. For example, grower or plant history may include, but is not limited to: whether the plant or seed set is an original breeding creation, a length of time the plant has been stewarded by the grower, an acquisition location of the original plant, etc. Morphologic and/or phenotypic descriptions or traits may include, but are not limited to: differences from or similarities to siblings, types of phenotypes (e.g., plant size (e.g., canopy height and width, etc.), flower (e.g., color, size, shape, THC/CBD content, oil content, etc.), growth profile (e.g., days to maturity, days to flower, etc.), fiber density, tensile strength, biofuel efficiency, phytoremediation use, nutritive potential, nutrient content, ionomics), range of phenotypes (e.g., high, medium, low), flower color (e.g., purple, white, orange, green, other), leaf shape (e.g., sativa-like or narrow lobed, mixed, indica-like or large lobed), leaf size (e.g., length, width, etc.), general plant structure (e.g., short, bushy, Christmas tree-like, tall, other), flowering window (e.g., days to flower), chemical profile (e.g., THC (high/med/low), CBD (high/med/low), terpenes, etc.), and the like.

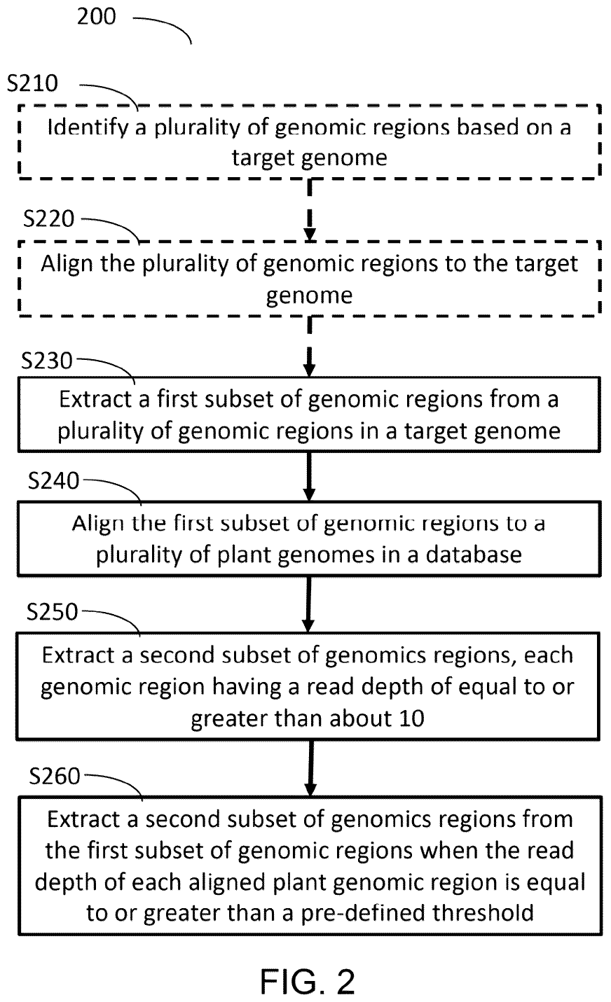

Turning to FIG. 2, one embodiment of a method 200 of genomic region identification may include: extracting a first subset of genomic regions from a plurality of genomic regions in a target genome at block S230; aligning a plurality of plant sequences in a database to the first subset of genomic regions at block S240; determining a read depth of each plant genomic region of the plurality of plant genomes that aligned with at least one of the first subset of genomic regions at block S250; and extracting a second subset of genomics regions from the first subset of genomic regions when the read depth of each aligned plant genomic region is equal to or greater than a predefined threshold at block S260. The method functions to determine genomic regions that are stable within a target genome (e.g., are present in a majority of cultivars or present in certain important groupings of cultivars to reflect certain population structure(s)) but that are also highly polymorphic. The method further serves as a basis to verify an identity of a cultivar, identify new cultivars, determine the uniqueness of a cultivar, verify supply chain integrity, map genetic traits, identify regions that may be targets for genetic manipulation or gene editing, conduct targeted selective breeding, implement predictive diagnostics, conduct synthetic compound production, and the like.

In some embodiments, the method 200 of FIG. 2 includes: extracting a first subset of genomic regions from a plurality of genomic regions in a target genome at block S230. Extracting may include annotating a first subset of genomic regions in the target genome, digitally extracting sequences from the target genomic data, and storing the sequences as well as its annotations in formats such as FASTA, GFF3, GTF, BED etc. In some embodiments, the first subset of genomic regions each can span, e.g., from about 100 bp to about 200 bp. The first subset of genomic regions can be extracted when the following criteria are met: (1) the region does not substantially align to a plurality of regions in the target genome; (2) the region does not substantially align to mitochondrial DNA or plastid DNA or other non-plant DNA; (3) the region has an annealing temperature or melting temperature in a range of about 55° C. to about 68° C.; (4) the region does not fall within a transposable element active area; and/or (5) the region does not contain highly repetitive DNA.

In some embodiments, the target genome is from a Cannabis including, but not restricted to, a public Cannabis cultivar genomes: Purple Kush, Finola, LA Confidential, Cannatonic, Pineapple Banana Bubba Kush, Jamaican Lion, Chemdog 91, and CBDRX (also known as Cs10), etc. In some embodiments, the target genome is from hemp. In some instances, the target genome is any plant genome, herb genome (e.g., lavender, rosemary, oregano, lemon pepper, thyme, purple passionflower, etc.), medicinal plant genome, agricultural crop genome, genomes for grape cultivars, genomes for hops cultivars, and the like.

In some embodiments, at least two genomic regions of the first subset of genomic regions may have overlapping genomic regions (i.e., tiled regions), such that the overlapping genomic regions may be combined into one genomic region. In such embodiments, identifying a plurality of genomic regions includes identifying neutral loci, non-neutral loci, putative genes of interest, orthologs, paralogs, features of interest, etc. across the target genome. Additionally, or alternatively, identifying may include using a plurality of chemical parameters, genetic structure features such as copy number variation, loci in Hardy-Weinberg Equilibrium, and the like.

The plurality of genomic regions may be identified by determining the number of reads aligned at a particular position or along a length of the target genome and determining which regions have a read depth greater than a predefined threshold. The predefined threshold may be a read depth of at least about 1, about 2, about 5, at least about 10, at least about 15, at least about 20, between about 5 to about 10, between about 5 to about 20, etc. In some instances, reads can overlap to achieve more depth in the sequencing. Alternatively, the plurality of genomic regions may be identified by dividing the target region (e.g., a loci, a portion of a chromosome, a chromosome, the genome) into regions comprising a predefined number of base pairs (bp) ranging from about 15 bp up to several thousand bp (e.g., about 25 bp, about 50 bp, about 75 bp, about 100 bp, about 250 bp, about 500 bp, about 1000 bp (1 kilo basepair (kb), 1.5 kb, 2 kb, 2.5 kb, etc.) such that the length is appropriate for the amplification methods being used.

In some embodiments, extracting the first set of genomic regions at block S230 further includes, optionally (shown by dashed line), aligning the plurality of genomic regions to the target genome at block S220. In such embodiments, aligning may utilize an algorithm or software application including, but are not limited to: BLAST, GeneWise, SFESA, LALIGN, VerAlign, and Lambda.

In some embodiments, the method 200 of FIG. 2 includes: aligning the first subset of sequences from a genomic region from, e.g., a Cannabis plant from an unknown cultivar, with a first subset of sequences of a corresponding genomic region from at least one known Cannabis cultivar in a database at block S240. Aligning tools may include, but are not limited to: BLAST, GeneWise, SFESA, LALIGN, VerAlign, and Lambda. In some embodiments, the plant genomes may be selected based on the data density in the database to maximize coverage and identify highly diverse or relevant regions. The database can include a library of plant samples that were created using sequencing techniques.

In some embodiments, the database may include as few as 2 sequenced cultivars (e.g., about 5, about 10, about 25, about 50, or about 75 sequence cultivars) or about 100 or more sequenced cultivars (e.g., greater than about 1,000, greater than about 2,000, greater than about 5,000, etc. sequenced cultivars). Sequencing of the cultivars in the database can have a depth of at least about 1×, at least about 2×, at least about 5×, at least about 10×, at least about 15×, at least about 20×, between about 5× to about 10×, between about 5× to about 20×, etc. In some embodiments, the cultivars in the database may have a breadth of genomic coverage of about 0.5%, about 1%, about 4%, about 10%, about 20%, about 50%, or about 100%. In some embodiments, the regions selected based upon database comparisons are not monomorphic and contain some level of polymorphism. The level of polymorphism includes, but is not limited to, a single bi-allelic SNP, multiple bi-allelic SNPs, a single multi-allelic SNP, multiple multi-allelic SNPs, INDELS (insertions or deletions), and other structural variants.

In some embodiments, the method to obtain the plurality of genomic regions may include, but are not limited to: whole genome sequencing, probe creation, targeted sequencing applications (e.g., using probes and/or primers), reduced representation sequencing methodology, qPCR, PCR, other amplification assays, other PCR assays including LAMP or loop-mediated isothermal amplification, probe enrichment for targeted sequencing approaches, multiplex marker assays, high level multiplex marker assays such as BioFire®, adaptive sampling targets using long read nanopore based sequencing technologies such as Oxford Nanopore Technologies®, and the like.

In some embodiments, the plurality of genomic regions for a plurality of plants within a database were prepared according to a genetic sequence barcoding or indexing process. Exemplary, non-limiting examples of such barcoding processes include: 2RAD, 3RAD, Illumina processes, Adapterama processes, and the like. Simply by way of example, the following two publications describe such processes: Glenn et al., 2019 “Adapterama I: universal stubs and primers for 384 unique dual-indexed or 147,456 combinatorially-indexed Illumina libraries (iTru & iNext),” Peer J., 7: e7755; and Glenn et al., 2019, “Adapterama III: Quadruple-indexed, double/triple-enzyme RADseq libraries (2RAD/3RAD),” Peer J., doi: 10.7717/peerj.7724.

Alternatively, or additionally, the plurality of genomic regions for a plurality of plants within a database can be prepared according to Illumina® iTru library preparation methods and standards, Illumina® iNext library preparation methods and standards, Daicel Arbor Biosciences preparation methods and standards, Pacific Biosciences® sequencing methods and standards, Oxford Nanopore Technologies® sequencing methods and standards, Hi-C (Arima Genomics) sequencing methods and standards, or the like.

In some embodiments, the method 200 of FIG. 2 includes determining a read depth of each plant genomic region of the plurality of plant genomes that aligned with at least one of the first subset of genomic regions at block S250; and extracting the second subset of genomic regions from the first subset of genomic regions when the read depth of each aligned plant genomic region is equal to or greater than a predefined threshold at block S260. The predefined threshold may be a read depth of greater than about 1%, greater than about 2%, greater than about 5%, greater than about 10%, greater than about 15%, greater than about 20%, between about 5% to about 50%, between about 5% to about 100%, etc. The second subset of genomic regions may be about 10 regions to about 3,000 regions or more, about 200 regions to about 400 regions, about 2,000 regions to about 3,000 regions, etc. depending on the size of the genome, structure of the genome, the diversity in the genome (among various cultivars, species, etc.), etc. In some embodiments, the second subset of genomic regions may be from 10,000 up to more than 250,000 regions depending on the size of the genome, structure of the genome, the diversity in the genome (among various cultivars, species, etc.), etc.

In some embodiments, the method 200 of FIG. 2 may optionally include using genotype likelihood estimations to extract the second subset of genomic regions from the first subset of genomic regions at block S250. The second subset of genomic regions may be about 1 or 2 regions, about 10 regions to about 3,000 regions, about 200 regions to about 400 regions, about 2,000 regions to about 3,000 regions, etc. depending on the size of the genome, structure of the genome, the diversity in the genome (among various cultivars, species, etc.), etc. In such embodiments, genotype likelihood may comprise estimations using an algorithm or software application including, but not limited to: ANGSD, ATLAS, MAPGD, VCFLIB, NGSTOOLS, PCANGSD, BASEVAR, EBG, FREEBAYES, GATK, REVEEL, SKMER, LOSTRUCT, ENTROPY, EVALADMIX, NGSADMIX, OHANA, SNPTEST, GUS-LD, POPLD, NGSRELATE, ALPHAASSIGN, WHODAD, BCFTOOLS ROH, LEP-MAP3, HETEROZYGOSITY-EM, SVGEM, HMMPLOIDY, BEAGLE, LB-IMPUTE, LINKIMPUT, LOIMPUTE, NOISYMPUTER, STITCH, etc.

In some embodiments, the method 200 includes stratifying or ranking the second subset of genomic regions based on the read depth (e.g., stored in database associated with a corresponding genomic region) of the aligned plant genomic regions. For example, genomic regions having a higher read depth may be ranked higher while genomic regions having a lower read depth may be ranked lower. Such read depth may be relative to a predefined threshold. The predefined threshold may be those regions that are greater than or equal to about 1, greater than or equal to about 2, greater than or equal to about 5, greater than equal to about 10, between about 5 and about 50, etc. such that the first subset of genomic regions that meet or exceed this predefined threshold is extracted to yield the second subset of genomic regions. One of skill in the art will appreciate that any predefined threshold may be used, tailored for a specific process or plant species or cultivar. In some instances, a lower threshold may be sufficient, while in other cases, a higher threshold may prove more useful.

### Confirmation of Plant Identity

Confirmation of plant identity encompasses two of the platforms described herein: supply chain certification and the double check test. Both the supply chain certification and the double check test start with extracting and sequencing genomic DNA from an “unknown” plant or plant tissue, and comparing the sequence information from the “unknown” plant to corresponding sequence information from one or more “known” reference plants (e.g., cs10 (aka CBDRx)). The sequence information from the one or more reference plants can be contained in a database or can be determined (e.g., concurrently) with the sequence information of the “unknown” plant.

In the double check test, the genotypic analysis can include an analysis of whether the “unknown” plant is a clone, sibling or distant relative to one or more of the reference plants. This analysis is based on pairwise differences; if the pairwise difference is below a specific threshold, the “unknown” plant and the reference plant are a clonal match, whereas if the pairwise difference is above a specific threshold, then the “unknown” plant and the reference plant are determined to be distant relatives. These thresholds were determined based on documented Cannabis sibling and clonal data.

The double check test and supply chain certification platforms can use comparisons to specific samples or groups of samples in a database in the genotypic analysis. As described herein, pairwise comparison can be used to determine the necessary threshold for the respective criteria.

In some embodiments, as shown in FIG. 3, a method 300 of verifying an identity of a plant or cultivar can include genotyping an unknown Cannabis plant or other plant sample at block S310; generating a genetic pattern specific to the unknown Cannabis plant or other plant sample based on a predefined set of genomic regions at block S320; comparing the genetic pattern specific of the unknown Cannabis plant or other plant sample to the genetic pattern from a reference Cannabis plant (e.g., cs10 (aka CBDRx)) at block S330; and outputting an indication of relatedness and/or matching between the genetic pattern of the reference Cannabis plant and the genetic pattern specific to the unknown Cannabis plant or other plant sample at block S340.

In some embodiments, the method 300 includes genotyping an unknown Cannabis plant or plant sample at block S310. In some embodiments, genotyping includes whole genome sequencing, reduced representation sequencing, restriction site associated DNA sequencing, double digest restriction site associated DNA sequencing, single restriction site associated DNA, double restriction site associated DNA sequencing, triple restriction site associated DNA sequencing, multiple restriction site associated DNA sequencing, amplicon sequencing, or the like. These methods also can include, but are not limited to, one or more of genomic DNA extraction, fragmentation of DNA using shearing or restriction enzyme digestion, adaptor ligation, limited cycle amplification, or combinations thereof.

In some embodiments, usually prior to sequencing, the genomic DNA may be processed using one or more size-exclusion techniques. For example, the genomic DNA may be processed to remove high molecular weight DNA (e.g., DNA greater than about 1000 bp in length, greater than about 5000 bp, greater than about 10000 bp, etc.), to remove low molecular weight DNA (e.g., DNA less than about 200 bp in length, less than about 100 bp, etc.), or a combination of both high and low molecular weight DNA size exclusion. The genomic DNA size exclusion may be performed with magnetic bead technologies, gel electrophoresis and subsequent purification, Pippin Prep, and the like. Alternatively, when ultra-long read sequencing platforms are used, no size-exclusion may be warranted.

In some embodiments, the method 300 includes generating a genetic pattern specific to the unknown Cannabis plant or other plant sample based on a predefined set of genomic regions at block S320. In some embodiments, the predefined set of genomic regions may be determined using the methods described in FIG. 2. In some embodiments, the predefined set of genomic regions may be based on genomic regions of interest, whole genomes, conserved regions, amplified regions, or the like. In some embodiments, the genetic pattern may comprise a genetic variation, for example specific single nucleotide polymorphisms (SNPs), short INDELs (insertions and/or deletions in genomic DNA), structural variations, duplications, inversions, etc., that are present in the unknown Cannabis plant or other plant sample (determined using the predefined set of genomic regions), particular read depths for each of the predefined set of genomic regions in the unknown Cannabis plant or other plant sample, and/or the presence or absence of at least a portion of the predefined set of genomic regions. In some embodiments, genomic regions may be identified using other methods, such as genetic mapping, trait mapping, gene validation, identification of regulatory elements, allelic variants (based on sequencing), gene expression levels, methylation patterns, proximity to structural elements, etc.

In some embodiments, a genetic pattern for a given cultivar is created by extracting physical sequences for the cultivar that correspond to the regions that were amplified by the predefined set of genomic regions (e.g., amplified using probes based on these predefined regions), and, optionally, concatenating the regions together for ease of sequence and/or fewer sequencing reactions.

In some embodiments, the method 300 includes comparing the genetic pattern specific to the unknown Cannabis plant or other plant sample to the genetic pattern at the corresponding region in the genome from a reference Cannabis plant (e.g., cs10 (aka CBDRx)) at block S330. A genetic pattern may include the sequence at each predefined region, some predefined regions, or a subset of the predefined regions, such that comparing includes comparing a sequence of the unknown Cannabis plant or other plant sample to a corresponding sequence in a known plant sample or a plurality of corresponding sequences of plant samples in a database. Each sequence may have one or more attributes, for example, a metric of diversity or heterozygosity, degree of matches at each base pair, degree of sequence similarity, a read depth, etc., as described herein. Comparing may additionally, or alternatively, comprise aligning the unknown cultivar sequence with the reference Cannabis cultivar and identifying regions that are mismatched (e.g., transversions, transitions, etc.) or missing (e.g., gaps).

In some embodiments, the method 300 includes outputting an indication of relatedness between the reference Cannabis plant or other plant genetic pattern and the genetic pattern specific to the unknown Cannabis plant at block S340. Such a method can be performed manually, using an automated platform, or combinations thereof. As described herein, a uniqueness, relatedness, heterozygosity, or genetic metric calculation can be used to determine whether the plant sample or cultivar sample has a clonal match, related match, or is not a match. A pairwise comparison is described herein, but other calculations may be similarly used as are known in the art. In one embodiment, a regional score may be calculated per region that is mismatched or missing based on the comparison. The regional score may represent the number of mismatches in the region. In some embodiments, all mismatches and gaps (or missing regions) are treated equally in the regional scoring; in some embodiments, all mismatches are treated equally while gaps are weighted; and in some embodiments, all gaps are treated equally while mismatches are weighted. In one exemplary, non-limiting embodiment, the weighting is 5× (e.g., a 3 bp gap has a score of −15) in the regional scoring, although other multipliers may be used (e.g., 2× to 10×, 3× to 6×, etc.).

Simply by way of example, each type of mismatch may be uniquely scored. For example, a transversion (A to T, A to C) may be given a first penalty (e.g., −2 for [[AA-TT; or AA-CC]]), a transition (A to G) may be given a second penalty (e.g., −0.5 for [[AA-GG]]), a gap may be given a third penalty (e.g., −2), and a homozygous state to a heterozygous state may be given a fourth penalty (e.g., −1 or −0.5, depending on whether the change was a transversion or transition). Alternatively, a score can be determined using pedigree analysis, clonal lineage analysis, or parentage analysis, etc.

There are a number of methods that can be used to determine familial relationships, and statistical analysis, if desired, can be performed on the results produced from any of such methods. The scores for all the genetic regions may be summed into an overall score, and then the overall score may be relativized by dividing the overall score by the total number of base pairs in each region. When the relativized overall score is less than a predefined threshold, then the unknown Cannabis cultivar is considered a match to the reference Cannabis cultivar, respectively. A predefined threshold to ascertain a sample as a clone or a relative to another sample is based on creating a distribution for all possible relatedness scores, defining confidence intervals around those scores, and considering what real scores are based on samples from a database with known familial relationships. Confidence intervals can be 95%, 99%, or 99.99%. For example, thresholds can be in the range of about 0 to about 0.0025 for clones and about 0.00251 to about 0.00341 for close relatives. Alternatively, depending upon the cultivar and, e.g., the evolutionary history of the cultivar, thresholds can be in the range of about 0 to about 0.05 for clones and about 0.051 to about 0.06 for close relatives.

### Cultivar Identification (ID) Testing

Cultivar ID testing can determine the phenotypic and genetic stability of a sample (e.g., for situations where a grower is evaluating which seeds to plant). That is, cultivars with low heterozygosity generally are more stable the subsequent generations, particularly upon selfing, than cultivars with high heterozygosity. Cultivar ID testing typically is based on the genetic similarity between the genome of the “unknown” plant and the reference genomes in the database (e.g., cs10 (aka CBDRx)). One of the difficulties is that Cannabis has a highly heterozygous genome. For example, when Cannabis, which is usually cross-pollinated, is selfed, highly heterozygous plants may exhibit inconsistent phenotypes for certain traits (e.g. yield, THC content, etc.). To address this issue, the methods described herein detect heterozygosity at specific sites in the genome by normalizing the count for heterozygous sites relative to an entire sequenced region (standardized to the number of sites that are included in the comparison minus heterozygous minimum divided by heterozygous range (difference between max and min)).

For cultivar ID testing, DNA samples are extracted from the plant and sequenced to identify specific markers. The sequence information then is compared to a database of sequences from Cannabis plants across cultivars in the species. Based on the results of the comparison, information can be provided regarding the identification of the cultivar for the plant tested, closely-related cultivars, least-related cultivars, and the copy number of genes involved in important agricultural traits like cannabinoid and terpene production. In some instances, the number of loci that are compared between the “unknown” plant and the one or more reference plants correlates with an increase in the accuracy of the genetic relationship that is established; in some instances, a single loci is sufficient to compare the “unknown” plant and the one or more reference plants and thereby identify the “unknown” plant.

Cultivar ID testing also can determine uniqueness. Uniqueness can be determined by producing a matrix of scoring across specific regions within the genome (e.g., using pairwise comparison) and calculating a degree of uniqueness based on Identity by State (IBS) (which is distinct from Identity by Decent (IBD)).

In another embodiment shown in FIG. 4, a method 400 of identifying a plant or cultivar can include genotyping an unknown Cannabis plant or other plant sample at block S410; generating a genetic pattern specific to the unknown Cannabis plant or other plant sample based on a predefined set of genomic regions at block S420; comparing the genetic pattern specific to a database of genetic patterns from known Cannabis plants at block S430; and outputting an identity or one or more attributes of the unknown Cannabis plant or other plant sample based on the comparison at block S440.

In some embodiments, the method 400 includes genotyping an unknown Cannabis plant and/or plant sample at block S410. In some embodiments, genotyping includes whole genome sequencing, reduced representation sequencing, restriction site associated DNA sequencing, double digest restriction site associated DNA sequencing, double restriction site associated DNA sequencing, triple restriction site associated DNA sequencing, amplicon sequencing, or the like. These methods may include, but are not limited to, genomic DNA extraction, fragmentation of DNA using shearing or restriction enzyme digestion, adaptor ligation, limited cycle amplification, or combinations thereof.

In some embodiments, the genomic DNA is further processed using one or more size-exclusion techniques. For example, the genomic DNA may be processed to remove high molecular weight DNA (e.g., DNA greater than about 1000 bp in length, greater than about 5000 bp, greater than about 10000 bp, etc.), to remove low molecular weight DNA (e.g., DNA less than about 200 bp in length, less than about 100 bp, etc.), or a combination of both high and low molecular weight DNA size exclusion. The genomic DNA size exclusion can be performed with magnetic bead technologies, gel electrophoresis and subsequent purification, Pippin Prep, and the like. Alternatively, when ultra-long read sequencing platforms are used, no size-exclusion may be warranted.

In some embodiments, the method 400 includes generating a genetic pattern specific to the unknown Cannabis plant or other plant sample based on a predefined set of genomic regions at block S420. The predefined set of genomic regions may be determined using the methods described in FIG. 2, FIG. 3, or other methods known to one of skill in the art.

In some embodiments, the method 400 includes comparing the genetic pattern specific to a database of known Cannabis plants at block S430. A genetic pattern may include the sequence at each predefined region, some predefined regions, or a subset of predefined regions, such that comparing includes comparing each sequence of the unknown Cannabis plant to a plurality of corresponding sequences of plant samples in a database. The sequences may have one or more attributes, for example, a metric of diversity or heterozygosity; a genetic similarity or polymorphism; a read depth; a sequence quality; etc., as described elsewhere herein. Comparing may additionally, or alternatively, include aligning the unknown cultivar sequence with sequences from one or more cultivars or plants in the database and identifying regions that are mismatched (e.g., transversions, transitions, etc.) or contain insertions and/or deletions (e.g., gaps).

In some embodiments, the method 400 includes outputting an identity or one or more attributes of the unknown Cannabis plant or other plant sample based on the comparison at block S440. A pairwise comparison is described herein, but other methods are known in the art. In one embodiment, a regional score may be calculated for each region that is mismatched or missing based on the above comparison. The regional score may represent the number of mismatches in the region. In some embodiments, all mismatches and gaps (or missing regions) are treated equally in the regional scoring; in some embodiments, all mismatches are treated equally while gaps are weighted; while in some embodiments, all gaps are treated equally while all mismatches are weighted. In one exemplary, non-limiting embodiment, the weighting is 5× (e.g., a 3 bp gap has a score of −15) in the regional scoring, although other multipliers may be used (e.g., 2× to 10×, 3× to 6×, etc.).

In some embodiments, each type of mismatch may be uniquely scored. For example, a transversion (A to T, A to C) may be given a first penalty (e.g., −2 for [[AA to TT; or AA to CC]]), a transition (A to G) may be given a second penalty (e.g., −0.5 for [[AA to GG]]), a gap may be given a third penalty (e.g., −2), and a homozygous state to a heterozygous state may be given a fourth penalty (e.g., −1 or −0.5 depending on whether it was a transversion or transition).

The scores for the regions may be summed into an overall score, and then the overall score may be relativized by dividing the overall score by the total number of base pairs in each region. When the relativized overall score is less than a predefined threshold, then the unknown Cannabis cultivar is considered a match to the reference Cannabis cultivar. As with the other methods described herein, these methods can be performed manually, using an automated platform, or combinations thereof.

### 2Dimensional (2D) Image Analysis

2D image analysis can be used to phenotype a Cannabis plant to identify the cultivar or as part of the phenotyping portion of the cultivar registration described above. Whole plant images and/or digital images of herbarium specimens can be used to provide information about leaf shape, powdery mildew detection, canopy shape, branching architecture, color, etc., using, for example, geometric morphometrics. The value of each attribute can be quantified and compared to a database of phenotypes for that attribute to determine where the “unknown” plant lies on the spectrum of species-level phenotypic trait data.

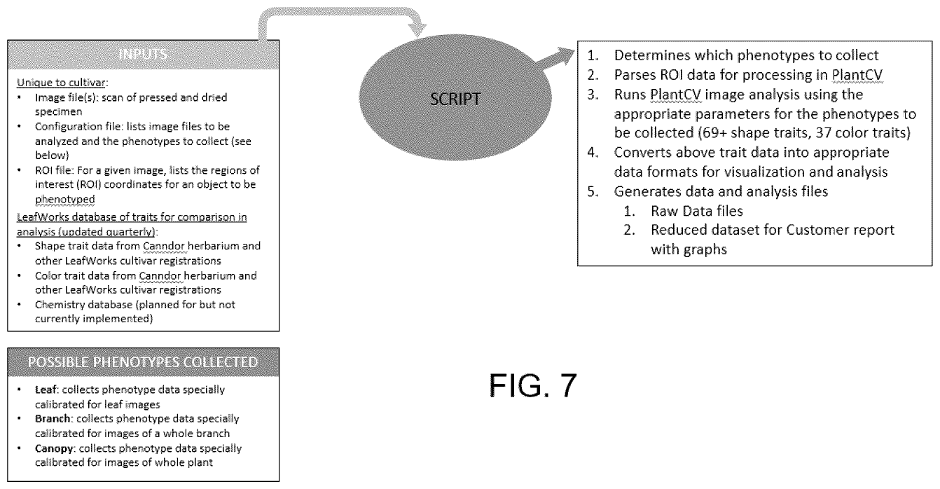

The PlantCV program can turn an image of a plant or plant part into a binary image (i.e., black and white), determine which pixels are different, and then determine features such as, without limitation, area, perimeter, height, width, aspect ratios, for different parts of the plant. For example, the PlantCV program can identify narrower leaf lobes, indicating sativa type, or wider leaf lobes, indicating indica type. The PlantCV program can identify, for example, leaves with thicker lobes, which can be an indication of air flow in the canopy and how much light gets through the canopy. Additionally or alternatively, the PlantCV program can identify a solidity trait, density of the leaf or tissue; ratio of the area; convex hull area; or combinations thereof, where a value of 1 indicates a solid object and a value less than 1 indicates an object having irregular boundaries or containing holes. Quantified traits can be compared to a database of images to understand the metric and provide an indication of a traits value and status. FIG. 7 shows a flow chart of the steps performed by the software, including inputs and outputs. Briefly, regions of interest (ROI) can be detected, manually or via an automated or machine learning processor, for objects in the image to be phenotyped, and a report generated.

PlantCV or other software such as, e.g., OpenCV, ImageJ, or TensorFlow, can be used to improve data collection for the number of leaflets per leaf, branching structure, and/or canopy structure. Machine-learning can be used to further improve the identification and quantification of tissue types or tissue structures (e.g., floral/inflorescence structures, trichomes, disease identification). Automated detection can gather information from images that were not taken specifically to measure an object (e.g., a leaf), and can allow for the ability to count substructures (e.g., flowers, buds, etc.) in addition to determining shape and color traits. For phenotyping traits such as canopy shape, an image of a whole plant on a standard background, if available, is preferred.

Table 1 shows a number of traits, along with possible implications related to each group of traits.

TABLE 1
List of Exemplary Traits

| | Trait | |
|---|---|---|
| Trait | Category | Possible Implication |
| blue frequencies | color | At the foundation, color is a unique |
| green frequencies | color | trait to different Cannabis cultivars. |
| red frequencies | color | Recording the specific color of a |
| lightness frequencies | color | cultivar is of interest to breeders and |
| green-magenta | color | patent officers. While red, blue, and |
| frequencies | | green frequencies are most often used |
| blue-yellow frequencies | color | for quick and informative reference, |
| hue frequencies | color | using this entire dataset gives a more |
| saturation frequencies | color | holistic view of a cultivar's color. |
| value frequencies | color | These color traits also represent the |
| hue circular mean | color | dataset used in our machine learning |
| hue circular standard | color | identification of powdery mildew |
| deviation | | (only uses PlantCV software and is |
| hue median | color | not integrated into a larger analysis |
| | | pipeline). |
| | | Additionally, color data can indicate |
| | | reflectance patterns of a given |
| | | cultivar. Reflectance can be used as a |
| | | proxy to determine the health of a |
| | | plant/if it is diseased. There is also |
| | | an evolutionary relationship between |
| | | reflectance pattern and phylogenetic |
| | | relationships between species, so |
| | | there is a possibility unique |
| | | signatures may belay a cultivar and |
| | | aspects of its pedigree. |
| | | Color traits are particularly useful in |
| | | the development of inflorescence/bud |
| | | trait quantification and trichomes. |
| top landmark coordinates | landmark | Landmarks are x, y coordinates added |
| bottom landmark | landmark | by the program to map out important |
| coordinates | | regions of the shape (plant) that can |
| center vertical landmark | landmark | be used to measure aspects of the |
| coordinates | | shape. The software can use |
| left landmark coordinates | landmark | landmarks to measure distances or |
| right landmark coordinates | landmark | relay important information about |
| center horizontal | landmark | canopy structure and plant |
| landmark coordinates | | architecture, which has implications |
| | | in best ag practices such as spacing in |
| | | the field, ability of light to penetrate |
| | | through the canopy, or airflow |
| | | through the canopy. |
| whether the plant | shape | These are basic geometric |
| goes out of bounds | | morphometric shape traits. Traits |
| area* | shape | with a * primarily are used to |
| convex hull area* | shape | determine the size and length/width |
| solidity • | shape | of shape objects (leaves, branches, or |
| perimeter | shape | canopy). Traits with a • are primarily |
| width* | shape | used to determine how closely a |
| height* | shape | shape resembles a circle vs how |
| longest path* | shape | many holes or gaps exist within an |
| center of mass • | shape | ellipse, which provides quantification |
| convex hull vertices • | shape | for how densely lobed leaves are, if a |
| object in frame | shape | leaf's leaflets are skinny or thick, |
| ellipse center • | shape | how airy or dense the canopy is, etc. |
| ellipse major axis length*• | shape | ‘Estimated object count’ was |
| ellipse minor axis length*• | shape | designed to count the number of |
| ellipse major axis angle*• | shape | leaflets for a given leaf. Traits |
| ellipse eccentricity • | shape | without demarcation are ways to |
| estimated object count | shape | determine if the program is collecting |
| | | data correctly. Size, shape, and |
| | | density characteristics are gateways |
| | | to important agricultural traits - e.g., |
| | | how large individual plants or |
| | | cultivars are, how that affects spacing |
| | | in the field, how much light can |
| | | penetrate the canopy, or the amount |
| | | of biomass that is above the surface. |

Turning now to various methods for phenotyping a Cannabis cultivar, in some embodiments, a method 800 of phenotyping a Cannabis cultivar can be performed by a processor. The instructions, executable by the processor, can be stored on a computer-readable medium. The method 800 can include receiving an input image of the Cannabis cultivar or an input sensor signal (the input signal being converted to an electrical signal that can be converted to an image) at block S810; identifying a plurality of regions of interest in the input image at block S820 (various embodiments are shown in FIGS. 5A-5C); identifying one or more traits (see Table 1) in one or more of the plurality of regions of interest at block S830; comparing the one or more traits to a database of known Cannabis cultivars, such that the database is configured to link each trait to a property of the Cannabis cultivar at block S840 (various embodiments are shown in Table 1); and outputting an indication of one or both of the property and the one or more traits at block S850.

In some embodiments, the method 800 includes receiving an input image of the Cannabis cultivar at block S810. The images can be captured by a computing device, image sensor, digital camera, or by any lenses paired with imaging acquisition software. The images can be transmitted to and received by a processor (e.g., via an antenna, transceiver, coil, etc.) configured to run a phenotypic analysis on the received image. The processor can be a part of the computing device that includes the imaging sensor or a remote computing device, for example, a remote server or the like. Alternatively, or additionally, the processor may be communicatively coupled to an image sensor (e.g., via a databus, antenna, coil, etc.) such that the processor is configured to receive an input sensor signal, which is converted to electrical signals followed by an image.

In some embodiments, a method 800 includes identifying a plurality of regions of interest in the input image at block S820. The regions of interest comprise various architectural or phenotypic properties of the plant (e.g., leaf structure, canopy structure, branching structure, etc.). FIGS. 5A-5C show various regions of interest of plant architecture. Turning to FIG. 5A, a region of interest can include canopy structure A. Canopy structure A refers to the overall spatial distribution of the above-ground portion of the plant of interest. Another region of interest may include height B, which refers to an average natural height at a predefined maturity. Height B refers to the height of the above-ground part of the plant. Another region of interest is an average spread C, which refers to the width of the above-ground part of the plant at a predefined maturity.

FIG. 5B shows various regions of interest related to leaf shape. Average area D refers to the average leaf area. Average perimeter E refers to the average leaf perimeter. Average number of leaflets F refers to the average number of leaflets from a single compound leaf. Average leaf width G refers to the widest distance perpendicular to the leaf major axis, which is the leaf width. Average leaf length H refers to the distance between the tip of the central leaflet to the node, including the length of the petiole, which is the leaf length. Leaf serration I refers to the edge structure of the leaf margin. Average leaf solidity J refers to the ratio between the leaf area and the convex hull where the convex hull is a polygon that bounds the leaf. Average central leaflet length K refers to the distance between the tip of the middle leaflet to the petiolar junction. Average central leaflet width L refers to the widest distance perpendicular to the main vein of the central leaflet. Average number of teeth of central leaflet M refers to the number of saw-like projections at the edge of the central leaflet (i.e., the leaf margin).

FIG. 5C shows various regions of interest related to reproductive yield. Average number of buds per inflorescence N refers to the number of floral clusters in each inflorescence. Average length of cola 0 refers to the distance between the tip of the apical inflorescence to the peduncle. Average width of cola P refers to the maximum distance of the cola that is perpendicular to the length of the cola.

In some embodiments, a method 800 includes identifying one or more traits in one or more of the plurality of regions of interest at block S830. In some embodiments, regions of interest may be used to identify one or more traits, which may include, but are not limited to, main stem diameter, presence or absence of mainstem grooves, color (qualitative range), pubescence (qualitative description), hollowness (qualitative description), average length between internodes (i.e., branching points, including leaves), canopy structure, average natural height at maturity, average spread at maturity, average leaf area, average leaf perimeter, average number of leaflets, average leaf width, average leaf length, leaf serration features, average leaf solidity, average central leaflet length, average central leaflet width, average number of teeth of central leaflet, average number of buds per inflorescence, average length of cola, or average width of cola. Alternatively, or additionally (and as described herein), one or more of a plurality of genomic regions can be used to identify one or more traits. The genomic regions can correlate to, track with, give rise to, or otherwise indicate or predict one or more traits.

In some embodiments, a method 800 includes comparing one or more traits to a database of known Cannabis cultivars, such that the database is configured to link each trait to a property of a Cannabis cultivar at block S840. In some embodiments, phenotypic traits are collected (e.g., determined, measured, etc.) manually; in some embodiments, phenotypic traits are collected automatically (e.g., electronically, digitally).

In some embodiments, a method 800 includes outputting an indication of the one or more properties and the one or more traits at block S850. In some embodiments, the output includes a color trait. Color traits can include, but are not limited to, blue frequencies, green frequencies, red frequencies, lightness frequencies, green-magenta frequencies, blue-yellow frequencies, hue frequencies, saturation frequencies, value frequencies, hue circular mean, hue circular standard deviation, and hue median.

Color is a unique trait to different Cannabis cultivars. A color trait can be indicative of various Cannabis cultivar properties. In general, a color trait can be indicative of a diseased state or a healthy state, for example infection due to powdery mildew, or the like. More particularly, color traits can be indicative of reflectance pattern properties of a given cultivar which can be used to determine the health of a plant. Further, a color trait can be indicative of light treatments, temperature treatments, and/or general stress, (e.g., drought stress, nutrient stress, etc.). Further, a color trait can be indicative of a phylogenetic property of a given cultivar. There can be an evolutionary relationship between reflectance pattern and phylogenetic relationships between species, so a unique color trait signature can convey aspects of a cultivar's pedigree. Still further, color traits can be indicative of properties related to inflorescence, bud development, and trichome quantification.

In some embodiments, the output can include a landmark trait and/or a shape trait. Landmark traits are x,y coordinates used by a processor to determine attributes like canopy structure. Landmark traits include, but are not limited to, top landmark coordinates, bottom landmark coordinates, center vertical landmark coordinates, left landmark coordinates, right landmark coordinates, and center horizontal landmark coordinates. Shape traits can include, but are not limited to, whether the plant goes out of bounds (e.g., may include output to a user to reimage or redraw the plant of interest), area, convex hull area, solidity, perimeter, width, height, longest path, center of mass, convex hull vertices, ellipse center, ellipse major axis length, ellipse minor axis length, ellipse major axis angle, ellipse eccentricity (e.g., how closely a shape resembles a circle vs how many holes or gaps exist within an ellipse, estimated object count (e.g., number of leaflets for a given leaf), the size and length/width of shape objects (leaves, branches, or canopy), how densely lobed leaves are, leaf thickness, and a density or airiness of a canopy.

The processor can use the coordinates to determine landmark traits, for example, sizes of, or distances between, physical plant features (leaves, stem, etc.) and canopy structure. Such landmark traits and shape traits can be indicative of agricultural practices important for a given cultivar, for example, spacing in the field, ability of light to penetrate through the canopy, airflow through the canopy, amount of biomass that is above the surface. In addition, leaf shape can be used to determine relatedness to other cultivars.

Traits can also include, but are not limited to, aerial architecture (e.g., branching structure, leaf arrangement), stem structure, node structure, extrapetiolar stipules structure, leaves structure (e.g., abaxial and adaxial surfaces, margin characters, leaflet blade characters), flower structure, perianth structure, inflorescences structure (e.g., arrangement, density), fruit yield, vegetative yield, etc.

Additionally, or alternatively, various traits (e.g., color, landmark, shape, etc.) can be linked to, or indicative of, vegetative yield, seed color, seed size, seed marbling, seed weight, morphological properties, medicinal uses, olfactory characteristics, chemical composition (e.g., terpenoids, cannabinoids, flavonoids, omega fatty acids, etc.), processing categories (fresh flower, extraction, hash, etc.), disease resistance, disease susceptibility, likelihood of being hermaphroditic, proportion of male seeds, proportion of female seeds, yield, agricultural output, industrial use properties, etc. Further, a leaf surface area parameter can correspond to a vegetative yield.

In some embodiments, phenotypic properties and genotypic properties can be combined, or phenotypic or genotypic properties can be used separately to determine one or more traits or properties of a cultivar, an ancestry of a cultivar (e.g., synapomorphies), a disease resistance or susceptibility of a cultivar, medicinal properties of the cultivar, for genetic mapping of traits of interest, prediction of phenotypes from biomarkers, etc. For example, one or more portions or steps of the methods of FIGS. 3, 4, and 6 can be combined to elucidate various characteristics or to identify or verify a cultivar or to link a trait to a property or identify or characterize genes of interest for traits of interest.

The systems and methods of the embodiments described herein, as well as variations thereof, can be embodied and/or implemented, at least in part, as a machine configured to receive a computer-readable medium storing computer-readable instructions. The instructions are executed by computer-executable components that can be integrated with the system and one or more portions of the processor on the computing device. The computer-readable medium can be stored on any suitable computer-readable media such as RAMs, ROMs, flash memory, EEPROMs, optical devices (e.g., CD or DVD), hard drives, floppy drives, or any suitable device, for example, on a remote server system (e.g., a cloud) or repository. The computer-executable component can be a general or application-specific processor, but any suitable dedicated hardware or hardware/firmware combination can alternatively or additionally execute the instructions.

The output (e.g., a report) from any of the methods described herein can provide actionable items for an individual or entity requesting and/or receiving the information (e.g., a “requester” of the information or a “recipient” of the report). It would be appreciated that a requester and a recipient can be the same individual or entity or different individuals or entities. A requester and/or recipient can include, without limitation, grower, farmer, cultivator, a government agency, a regulatory agency, a dispensary, an individual, law enforcement, a researcher, a company, etc. For example, actionable data includes the quantified nature of traits being measured (e.g., leaf shape, color, powdery mildew detection, canopy shape, branching architecture) so a grower can know if a plant meets their specifications or if a breeder has more work to do to either develop or stabilize a trait. Outputs also can be used to evaluate environmental effects on genotype.

As touched on above, machine learning can be used in conjunction with any of the platforms described herein in the genotypic analysis (e.g., to link phenotypes to genomic regions or markers or to predict phenotypes based on, for example, molecular markers, gene expression, etc.) or in the phenotypic analyses (e.g., to automate aspects of the existing pipeline (for ROI detection, object/structure detection) or to identify features of plants in non-staged environments (e.g., images taken without a white uniform backdrop or at a pre-determined distance for calibration)).

### Exemplary Applications of Methods Described Herein

Double check test—tissue culture company is producing plants and wants to verify that they have used the correct cultivar in their collection and are not creating plants of the wrong registered variety.

Double check test—cultivator harvests 4 batches of plants but the labels get mixed up. They use references they know are cultivar 1, 2, 3, and 4 and then we compare the unknown batches to the known references to sort out the mixed up cultivars.

Cultivar registration—breeder has created a new cultivar and wants to 1) characterize it genotypically and phenotypically for a PVP Certificate and to register the material in a database to stake their claim in the market with an auditable reference if they feel that people are using their plant material out of contract terms.

Supply chain certification—a brand wants to prove that their product is of a single cultivar source. They submit the reference and each batch created is then tested to show it matches the cultivar that it is supposed to be and that no adulteration is present.

Phenotyping—a breeder has created a new cultivar and wants to understand how its physical features measure up to the rest of the species. They perform image analysis to understand how it compares.

Cultivar registration—a region of cultivation wants to apply for appellation status and needs to show that the same genetics have better output in their region vs others. They use the genetic analysis to confirm it is the same material and the phenotyping analysis to show higher quality in their region.

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated invention(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

In accordance with the present invention, there may be employed molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Genomics Database

The genomics database contains sequences of over 5000 samples of diverse Cannabis accessions that were collected from different regions worldwide. These accessions cover almost the entire genetic diversity in Cannabis. In addition, Cannabis cultivars from known clonal groups and familial relationships are present in the database.

The database was created by collecting the samples, extracting and sequencing the DNA from those samples, and eventually fingerprinting genomic variation across them. The genomic variation has been identified to uniformly cover the entire Cannabis genome. A stepwise process to establish the LeafWorks genomics database is presented below:
1. Sample Collection—Samples collected from Cannabis plants, including a voucher for a physical plant paired to genetic data whenever possible.
2. DNA Extraction, Probe Design and Sequencing
   I. DNA Extraction—DNA was extracted from tissue samples using either a standard CTAB procedure or using a Qiagen DNeasy Plant Mini kit. Extracted DNA was quantified using a Thermo Scientific Nanodrop 2000c. If needed, samples were further purified using Speedmag beads and quantified again. Finally, DNA samples were standardized to 20 ng/μ0.1 concentration in TE buffer.
   II. Probe design—To optimize the throughput and accuracy of sample DNA fingerprinting for different services, we identified specific regions across the Cannabis genome for capturing genome-wide polymorphisms. A stepwise approach was taken to identify the regions of interest, which were used for target sequence capture and variant discovery in diverse Cannabis samples. In brief, we first sequenced a subset of over 1000 samples using the 3RAD reduced representation library preparation method. These 3RAD sequences were used to identify polymorphic loci in the sampled individuals using the STACKS software (catchenlab.life.illinois.edu/stacks/manual/#intro). Finally, a subset of the loci were extracted using different parameters and used for the probe design. The detailed process of probe design is as follows: Stacks identifies loci in a set of individuals, either de novo or aligned to a reference genome (including gapped alignments), and then genotypes each locus. Stacks incorporates a maximum likelihood statistical model to identify sequence polymorphisms and distinguish them from sequencing errors. Stacks employs a Catalog to record all loci identified in a population and matches individuals to that Catalog to determine which haplotype alleles are present at every locus in each individual.
      A. 3RAD sequencing of a subset of samples—For library preparation, we follow the methodology of Illumina library preparation using a 3RAD Adapterama reduced representation design. Briefly, DNA samples were digested with restriction enzymes. Standard restriction enzymes used were NheI, EcoRI, and XbaI. Digested gDNA was size selected using Speedmag beads to optimize for digested gDNA fragments between 200-500 base pairs (bps) in size. Next, to multiplex the samples, inner barcodes were ligated onto the digested gDNA, followed by adding outer barcodes to the libraries using PCR. The 3RAD libraries were sequenced using Illumina HiSeq 3000 platform to obtain 150 bps paired-end sequencing reads.
      B. Identification of loci for probe design—The 3RAD sequencing data was analyzed using the STACKS program (catchenlablife.illinois.edu/stacks/manual/#intro), which is specifically developed to analyze the restriction-enzyme based sequencing datasets. The sequences obtained from 3RAD libraries were demultiplexed and quality filtered (details of which are described herein) with the sample-specific barcodes using the “process radtags” plugin in STACKs with “-P, -c, -q, -r, -inline_inline, -renz_1, -renz_2” parameters. Afterwards, the sample-specific reads were quality filtered using default parameters in the Trimmomatic software (Bolger et al., 2014). The sample-specific reads were used to build and catalog loci (denovo or through genome alignments) using the “ustacks” and “cstacks” STACK plugins, which were genotyped across all the samples in the 3RAD dataset with “gstack” plugin. We further screened the STACKs loci to identify potentially useful genomic regions for probe design using three different metrics—(a) sites that were deemed highly polymorphic, (b) regions of known genes of interest (GOI), (c) regions randomly spaced across each chromosome on the Cannabis genome. A second filtering step was imposed to remove regions that show matches to (1) Cannabis mitochondria/plastid genomes (2) other non-plant DNA (3) multiple regions on the Cannabis genome, (4) fall within any transposable element active area, and/or (5) the regions containing highly repetitive DNA. Also, the target regions that most likely hybridize at Tm=55-65° C. were retained. The remaining regions were used as a reference to re-align all the samples and the regions covering 99.9% samples at a read depth of >10 were kept for probe design and targeted sequencing of all the samples in the database. These probe regions represent highly polymorphic regions as well as the most stable regions (e.g., are present in the majority of cultivar genomes) across the Cannabis genome.
3. Building Genomics Database
   I. Probe Library Preparation and Sequencing—Leaf tissue from all the Cannabis accessions in the database were used to extract DNA for library preparation. In brief, DNA is extracted from tissue using either a standard CTAB procedure or using a Qiagen DNeasy Plant Mini kit. Extracted DNA is then quantified using a Thermo Scientific Nanodrop 2000c. If needed, samples are further purified using Speedmag beads and quantified again. DNA samples are then standardized to 20 ng/11.1 concentration in TE buffer.
   II. For library preparation, we follow the methodology of Illumina library preparation using a 3RAD Adapterama reduced representation design. Briefly, DNA samples are first digested with restriction enzymes. Standard restriction enzymes used are Nhe I, EcoRI, and Xba I. Digested gDNA is size selected using Speedmag beads to optimize for digested gDNA that is 200-500 bp in size. Next, inner barcodes are ligated onto digested gDNA. Afterwards, outer barcodes are added onto libraries using PCR. These libraries are then cleaned using Speedmag beads. The cleaned 3RAD libraries are then hybridized to probes using sequence capture protocols developed by Arbor Biosciences. The probes allow for 3RAD libraries to be enriched targeted loci for sequencing where these captured probes are PCR amplified at the end of hybridization. The libraries were sequenced using Illumina NovaSeq or MiSeq platforms to obtain 150 bp paired-end sequence reads.
   III. Read Sequence Processing, Alignments—The probe library sequences were demultiplexed and using the sample-specific barcodes with the “process radtags” plugin in STACKS with “-P, -c, -q, -r, -inline_inline, -renz_1, -renz_2” parameters. Afterwards, the sample-specific reads were quality filtered to trim adapters and filter out low quality sequence reads using default parameters in the Trimmomatic software (Bolger et al., 2014). The resulting high-quality reads were aligned against the reference Cannabis genome (cs10 (aka CBDRx)) with minimap2 software (Li et al., 2018) using the default short read parameters. The sample-specific alignment files were converted to binary alignment format (BAM), sorted, and indexed for further processing. The BAM files were also processed to mark PCR duplicates using the “MarkDuplicates” plugin in PICARD Tools (broadinstitute.github.io/picard/).
   IV. Variant Discovery and Filtering—Genotype-specific variant call format (gVCF) files for each sample were obtained from the processed BAM files using the “HaplotypeCaller” plugin in the Genome Analysis Toolkit (GATK) software (gatk.broadinstitute.org/hc/en-us). The gVCF files were combined to build a database of all the samples using the “GenomicsDBlmport” plugin in GATK. The resulting database was genotyped using the GATK's “genotypeGVCF” plugin to obtain the polymorphic loci in a VCF format. These polymorphic loci were further filtered to retain loci meeting following criteria: (1) More than 50% samples have minimum read depth of 10 at the individual loci, (2) Average read depth>10, (3) maximum missing data<10%, and/or (4) Minor allele frequency<0.05.
4. Genomics Database—After filtering, at the time of probe creation, the Genomics Database consisted of about 1505 samples and 10,105 high quality, polymorphic loci distributed across the entire Cannabis genome.

### Example 2—Double Check Test

Double check test takes in a Cannabis sample from a sample provider and tests its match against a known (potentially same cultivar as the provided sample) sample in the database. After receiving the sample, it is processed as follows to prepare and deliver a double check test report back to the sample provider:
A. DNA Extraction, Library Preparation, and Sequencing—When received by the lab, plant tissue is processed for DNA sequencing. In brief, DNA is extracted from tissue using either a standard CTAB procedure or using a Qiagen DNeasy Plant Mini kit. Extracted DNA is then quantified using a Thermo Scientific Nanodrop 2000c. If needed, samples are further purified using Speedmag beads and quantified again. DNA samples are then standardized to 20 ng/μl concentration in the TE buffer. A modified library preparation method has been used to prepare libraries for probe regions. This approach is the same approach to library preparation that was used to generate the database. The details are as follows:
B. For library preparation, we follow the methodology of Illumina library preparation using a 3RAD Adapterama reduced representation design. Briefly, DNA samples are first digested with restriction enzymes. Standard restriction enzymes used are Nhe I, EcoRI, and Xba I. Digested gDNA is size selected using Speedmag beads to optimize for digested gDNA that is 200-500 bp in size. Next, inner barcodes are ligated onto digested gDNA. Afterwards, outer barcodes are added onto libraries using PCR. These libraries are then cleaned using Speedmag beads. The cleaned 3RAD libraries are then hybridized to probes using sequence capture protocols developed by Arbor Biosciences. The probes allow for 3RAD libraries to be enriched targeted loci for sequencing where these captured probes are PCR amplified at the end of hybridization. The libraries were sequenced using Illumina NovaSeq or MiSeq platforms to obtain 150 bps long paired-end sequence reads.
C. Read Processing and Alignments—The read processing and variant discovery method is the same as described in Example 1. Basically, the probe library sequences were demultiplexed (if containing multiple samples) and using the sample-specific barcodes with the “process radtags” plugin in STACKS with “-P, -c, -q, -r, -inline_inline, -renz_1, -renz_2” parameters. Afterwards, the sample-specific reads were quality filtered to trim adapters and filter out low quality sequence reads using default parameters in the Trimmomatic software (Bolger et al., 2014). The resulting high-quality reads were aligned against the reference Cannabis genome with minimap2 software (Li et al., 2018) using the default short read parameters. The sample-specific alignment files were converted to binary alignment format (BAM), sorted, and indexed for further processing. The BAM files were also processed to mark PCR duplicates using the “MarkDuplicates” plugin in PICARD Tools (broadinstitute.github.io/picard/).
D. Variant Discovery and Sample Genotyping—Genotype-specific variant call format (gVCF) files for each sample were obtained from the processed BAM files using the “HaplotypeCaller” plugin in the Genome Analysis Toolkit (GATK) software (gatk.broadinstitute.org/hc/en-us). The gVCF files from double check test samples are merged with the gVCF files in the genomics database using the “GenomicsDBImport” plugin in GATK. The resulting database (database samples+double check samples) are genotyped using the GATK's “genotypeGVCF” plugin to obtain the polymorphic loci in a VCF format. The polymorphic loci selected in the previously defined genomics database are extracted to calculate relatedness between the double check sample against the desired samples in the genomics database.
E. Defining Match/No Match in Double Check Samples—A relatedness calculation can be used to determine whether the plant sample or cultivar sample has a clonal match, related match, or no-match. For example, for verifying an identity of a cultivar and/or verifying supply chain label claims, the comparison can be between a plant or cultivar sample and a specific sample or group of samples in a database (e.g., Cannabis samples cross-checked to a Cannabis cultivar in the database). To assess if the double check samples match to each other, we generate a pairwise matrix of relatedness scores based on the similarity in the nucleotide sequences between individuals within a pair. The samples were determined to be a match, if their pairwise relatedness score won't exceed the threshold scores established from a pairwise comparison of known clonal matches.

### Example 3—Supply Chain Certification

For supply chain certification, the methods are similar to the double check methods described in Example 2 but used in different applications. The need for product transparency and consistency is essential. The supply chain certification is a DNA-based test that tracks and verifies Cannabis samples as it moves along the supply chain. This verification service tracks samples, assesses batch consistency, identifies adulturation, incorporates DNA-level quality control measures, and mitigates fraud.

### Example 4—Cultivar Genetic Testing from the Cultivar Registration

Cultivar registration process implies a genetic fingerprinting and phenotypic characterization of various features that are unique to a specific Cannabis cultivar.
A. Genetic Fingerprinting of a Cultivar—This process involves extracting DNA, whole genome sequencing, and variant identification steps. However, once a VCF file of polymorphic loci (genomics database samples+cultivar registration sample) defined in the genomics database is generated, different population genomic metrics are calculated to obtain genomic signatures of the new cultivar against the genomic signatures of all other samples in the genomics database. Currently, a distribution of three metrics, heterozygosity, uniqueness and genetic distance, have been implemented to categorize new cultivar in relation to the database.
   I. Cultivar Heterozygosity Relative to Database Samples—As used herein, “heterozygosity” refers to an estimate of the degree of genetic variation within a plant sample relative to a database or a plurality of plant samples. Heterozygosity is calculated by either (1) normalizing the count for heterozygous sites to all the SNPs detected (standardized to the number of sites that are included in the comparison—heterozygous minimum and divided by heterozygous range (difference between max and min)); or (2) calculating the number of heterozygous states per sample (cultivar of interest) across all or a subset of the genomic sites in the database and plotting this against all or a subset of the samples in the database. Heterozygosity analysis can indicate the phenotypic and/or genetic stability of a plant or cultivar sample over generations. For example, samples with low heterozygosity will be more phenotypically stable than the ones with high heterozygosity in the subsequent generations. A histogram plot of the heterozygosity scores for the new cultivar relative to samples in the database can be included with the Cultivar Registration report.
   II. Determining Cultivar Uniqueness relative to Genomics Database Samples—As used herein, “uniqueness” or “relatedness” refers to how rare or common a plant is relative to other cultivars (for example, in a database). Uniqueness or relatedness refers to the metrics generated by calculating the Identity by State using a pairwise comparison between samples in a database to determine how similar two samples are based on their nucleotide sequence. The pairwise comparison may be further used to determine known or unknown clonal or familial relationships between samples. Uniqueness or relatedness may be calculated by performing a pairwise comparison at genomic regions or sites determined at all the selected loci in the genomics database, between a cultivar of interest to the unique genetic patterns for one or more, a plurality of, or all of the cultivars in the database. For example, it can determine if and how much the genotype of a cultivar is different or not compared to the genotype of other cultivars at any specific locus in a database with varying scoring rules (score threshold obtained from pairwise comparison of known clonal or familial relationships). Alternatively, uniqueness or relatedness may be calculated by comparing each loci for all the samples in the genomics database, any differences in each region are recorded as unique values. The values are then normalized by standardizing the number of sites that are included in the comparison, minimum value in the database, and/or the range (difference between max and min). A lower score means more relatedness (when compared between two samples) or less uniqueness (when compared to the database).
      Further for example, for registering a potentially new cultivar, the comparison may be used to determine what a plant or cultivar sample is most similar to or most dissimilar from relative to all the samples in the genomics database. For cultivar identification, the comparison can be used to determine what plant or cultivar sample is most similar to or most dissimilar from the other samples in the genomics database. A histogram plot of the relatedness scores for the new cultivar relative to samples in the genomics database can be included with the cultivar registration report.

### Example 5—Phenotypic Characterization/Cultivar Registration

Phenotypic characterization of Cannabis, including marijuana and hemp plants, utilizes herbarium vouchers as well as traits evaluated digitally and/or hand measured from living plants. Table 2 includes the list of phenotypic traits collected for the cultivar registration process. Additionally, the report incorporates interviews (e.g., with the requester) about the breeding history, pedigree, and cultivation of the cultivar (Table 3). Finally, requesters may volunteer to submit any cannabinoid or terpene data they have received from analysis that can be analyzed and incorporated into the final report.
a. Interviews with Requester—Upon beginning the cultivar registration process, a specialist conducts at least one interview with the requester to ascertain information about the cultivar. The list of questions each requester is asked are listed in Table 3. If a specialist does not collect data from living plants (see section below), the requester may be responsible for providing these phenotypes as well. If a requester has elected to submit their crop for cannabinoid and terpene analysis, they may submit this report for incorporation into the cultivar registration report.
b. Phenotypic Characterization of Living Plants—Traits relating to plant architecture and reproductive plant parts (Table 2) are measured from live plants just before or at the time of harvest. Photos and video are taken as well for reproductive measurements (Table 2), documentation, and characterization of cultivars. These traits are incorporated into the cultivar registration report.
c. Herbarium Voucher Creation—At least two herbarium vouchers can be collected for living plants of the cultivar getting registered. One voucher, for example, includes a minimum of three leaves for leaf shape measurements (Table 2), while the other voucher can be related to a branch from the plant with a large inflorescence represented. The vouchers can be used to identify the botanical description (Table 2). Additional vouchers can be obtained if desired to capture highly phenotypically variable cultivars. Briefly, herbarium vouchers are prepared by removing leaves and a branch from the living plant. Plant parts are arranged on 100% cotton blotting paper between two ventilators and stacked within a plant press, then compressed. The compressed plant press is left to dry in warm, dry conditions until the plant material is completely dry. Once dry, dried plant material is affixed to 100%, acid-free archival-grade herbarium mounting paper using a 30% dilution of University of Oregon-type glue, a polyvinyl acetate adhesive that is inert in long term storage. All herbarium vouchers are barcoded and cataloged in the Canndor Herbarium.
d. Phenotypic Characterization of Herbarium Vouchers—The majority of phenotypic traits in the cultivar registration report are measured from herbarium vouchers.
   i. Manually collected phenotypic data—Five leaf shape traits and the botanical scientific description are collected manually by a specialist at this time (Table 2).
   ii. Digitally collected phenotype data—All remaining phenotype data are collected digitally (Table 2). Herbarium vouchers collected from the cultivar are scanned using an Epson WorkForce DS-50000 Document Scanner at 400 dpi, in color, saved as a .png file format. Digital images of the herbarium vouchers are then analyzed using PlantCV (Fahlgren et al., 2015; Gehan et al. 2017), an open-source, community-developed computer vision software that is a series of image processing and normalization modules that can be designed to the users' needs. The cultivar registration pipeline builds upon this software to have a custom workflow to analyze Cannabis phenotypic diversity. Once image analysis is performed, images are compared to the database of phenotypic data to assess the phenotypic variability of a given cultivar.
      1. Data Preparation—PlantCV requires a user to input the region of interest (ROI) for analysis; i.e., coordinates drawn around plant material to be measured, such as a leaf or branch. These coordinates are analogous to the pixels of an image. The cultivar registration pipeline automates the input of these ROI and the type of analysis to be performed, which may vary if it is for a leaf, a branch, or plant canopy. ROI can be drawn as a rectangle (consists of X,Y coordinates, height, and width), circle (consists of X,Y coordinates and radius), or custom shape (consists of any number of X,Y coordinates that are connected by the program). An ROI file is created in a tab delimited file format with the ROI coordinates for analysis and their corresponding image file. A configuration file is also created in a tab delimited file format that records the images to be analyzed, the type of ROI that was drawn (i.e, rectangle, circle, or custom), and the type of plant material to analyze (i.e., leaf, branch, or canopy). These input files are used in the pipeline described below.
      2. Pipeline Design—The cultivar registration pipeline for phenotypic analysis consists of seven computer scripts performing image and data analysis using the programs PlantCV and R (R Core Team 2023), coordinated by a single wrapper script that automates the analysis.
         a. User Inputs—The cultivar registration scripts can use a specific subfolder structure within a computer processor. Four folders must be present: 1) a ‘bin’ containing all scripts for analysis, 2) a ‘local’ folder containing phenotypic traits that will be used in downstream data analysis, 3) a cultivar data folder, specific to each analysis which will contain all resulting analysis files, and 4) an ‘Inputs’ subfolder within the cultivar data folder that must be unique to each cultivar which contains all digital scans to be included in the analysis, the configuration file, and the ROI file (see above). If a requester provides their cultivation location (such as a farm or lab), the latitude and longitude is also included in the input folder. If a requester provides their cannabinoid and terpene report, these data are also prepared and included in the input folder.
         b. Pipeline—The following tasks are performed by the seven scripts within cultivar registration analysis pipeline: the wrapper script creates subfolders within the cultivar data folder created for data files (performed by the wrapper script); 1) input files are parsed and prepared into the necessary format for analysis; 2) PlantCV analysis of images in input folder; 3) statistical analyses and plots of leaf shape and leaf color variation performed in R, 4) climate data for the farm location are pulled from BioCLIM in R; 5) chemistry data from the customer are plotted in R; 6) python script to merge the cultivar data within the phenotype database; 7) genomics portion of the analysis is performed.
         i. PlantCV Script—Broadly, this script allows users to run PlantCV in a loop for a specified number of Images and ROIs. This script can be run on any image that is captured through a scan, android phone, iPhone, or DSLR camera. For each ROI, the traits listed as “digital” for collection method in Table 2 are collected. The steps of the PlantCV analysis in the LeafWorks pipeline are as follows:
         1. Preparing the Raw Image—Each image is taken in as an RGB image with channel “s”. The image is then thresholded with the following values; threshold=35; max value=255; object type=“light”. A median blur value of ksize=10 was used. A fill value of, size=200, and a mask color of “white”.
         2. Isolate the ROI and Identify Objects Within—Each ROI within an image is then pre-processed using plant contour, plant mask, and a hierarchy. This is done using the default parameters.
         3. Determine what Phenotypic Analysis to run based on Plant Material Type—Now isolated and preprocessed, color and shape traits are determined for each ROI based on the plant material type (leaf, branch, or canopy). For leaf and canopy images, shape traits are gathered by running the analyze objects analysis with default parameters; the Watershed segmentation with a value of 75; and pseudo landmarks with default parameters. For branch images, the shape is based on skeletonizing the image, with a prune size of 200. The rest of this analysis follows the protocol outlined here, at default parameters. For all plant material types, color was determined using the analyze color command.
         4. Outputs—This script will produce a folder for each image, named after that image. Within each of these image folders are the following:
         a. the original image file
         b. six intermediate images across the thresholding process
         c. a subfolder by plant material type. In these subfolders are the intermediary images per ROI showing shape and color processing and analysis.
         d. a quantitative trait table for each image, broken down by ROI. Shape data are collected as pixels. These files are what the plotting shape and color R script will use.
         ii. Plotting Leaf Shape and Color R script—This R script is designed to plot the shape and color traits based on the raw table per image generated from the PlantCV image analysis script. The dependencies of this program are: Tidyr, ggplot2, ggradar, ggcorrplot, ggrepel, ggbiplot, ggfortify, scales, data.table, reshape2, readxl. The inputs are fed in through the pipeline. Pixel measurements are converted into mm using the scale ratio of 0.0635. This script processes the complex format of the raw tables from the image analysis into a more manageable format for R, then creates a series of graphs and tables. This script also uses predetermined leaf color and shape values across the database to get a population mean.
         iii. Plotting Location Climate Data R Script—This R script takes the latitude and longitude coordinates provided by the requester and uses the R library “Raster” to generate worldclim or bioclim data at a resolution of 10. Given that worldclim data has a scale factor of 10 (i.e., Temp=−37, which is actually−3.7° C.) and has already been accounted for and converted to the correct values.
         1. Output values provided in the cultivar registration report to the requester:
         a. Annual Mean Temperature
         b. Max Temperature of Warmest Month
         c. Min Temperature of Coldest Month
         d. Temperature Annual Range
         e. Annual Precipitation
         f. Precipitation of Wettest Month
         g. Precipitation of Driest Month
         2. Additional output values:
         a. Latitude
         b. Longitude
         c. Mean Diurnal Range
         d. Isothermality
         e. Temperature Seasonality
         f. Mean Temperature of Wettest Quarter
         g. Mean Temperature of Driest Quarter
         h. Mean Temperature of Warmest Quarter
         i. Mean Temperature of Coldest Quarter
         j. Precipitation Seasonality
         k. Precipitation of Wettest Quarter
         l. Precipitation of Driest Quarter
         m. Precipitation of Warmest Quarter
         n. Precipitation of Coldest Quarter
         iv. Plotting Chemistry Data R script—If a requester elects to submit cannabinoid and terpene data for inclusion in the cultivar registration report, the data from the individual sample is entered by hand into a chemistry configuration file and then plotted. A database of chemistry data is also pulled from the local folder during the analysis pipeline for the graphs that compare the individual against a background. This database should be updated every quarter to present more accurate data. Seven Violin plots generated with this script, breaking the metabolites by groups. Each plot is similar in construction: for each trait, there is a background distribution and a yellow dot on that distribution, representing the individual sample being processed. If no data was provided (i.e., missing data) then “No Data Provided” is written on the background distribution of the plot.

TABLE 2
List of phenotypic traits collected for Cultivar Registration

| | | Plant | | |
|---|---|---|---|---|
| | Collection | Material | Trait | |
| Trait | Method | Measured | Category | Possible Implication |
| Blue frequencies | Digital | Leaf | Color | At the foundation, color is a unique trait to |
| | | voucher | | different Cannabis cultivars. Recording |
| Green frequencies | Digital | Leaf | Color | the specific color of a cultivar is of interest to |
| | | voucher | | breeders and patent officers. While red, blue, |
| Red frequencies | Digital | Leaf | Color | and green frequencies are most often used for |
| | | voucher | | quick and informative reference, using this |
| Lightness | Digital | Leaf | Color | entire dataset gives a more holistic view of a |
| frequencies | | voucher | | cultivar's color. These color traits also |
| Green-magenta | Digital | Leaf | Color | represent the dataset used in our machine |
| frequencies | | voucher | | learning identification of powdery mildew |
| Blue-yellow | Digital | Leaf | Color | (only uses PlantCV software, is not integrated |
| frequencies | | voucher | | into a larger analysis pipeline). |
| Hue frequencies | Digital | Leaf | Color | Additionally, color data can indicate |
| | | voucher | | reflectance patterns of a given cultivar. |
| Saturation | Digital | Leaf | Color | Reflectance can be used as a proxy to |
| frequencies | | voucher | | determine the health of a plant/if it is |
| Value frequencies | Digital | Leaf | Color | diseased. There is also an evolutionary |
| | | voucher | | relationship between reflectance pattern and |
| Hue circular mean | Digital | Leaf | Color | phylogenetic relationships between species, so |
| | | voucher | | there is a possibility unique signatures may |
| Hue circular | Digital | Leaf | Color | belay a cultivar and aspects of its pedigree. |
| standard deviation | | voucher | | Color traits will be particularly useful in the |
| Hue median | Digital | Leaf | Color | development of inflorescence/bud trait |
| | | voucher | | quantification and trichomes. |
| Top landmark | Digital | Leaf | Leaf | Landmarks are x, y coordinates added by the |
| coordinates | | voucher | landmark | program to map out important regions of the |
| Bottom landmark | Digital | Leaf | Leaf | shape (plant) that can be used to measure |
| coordinates | | voucher | landmark | aspects of the shape. The software can use |
| Center vertical | Digital | Leaf | Leaf | landmarks to measure distances or relay |
| landmark | | voucher | landmark | important information about canopy structure |
| coordinates | | | | or plant architecture, which has implications |
| Left landmark | Digital | Leaf | Leaf | in best ag practices such as spacing in the |
| coordinates | | voucher | landmark | field, ability of light to penetrate through the |
| Right landmark | Digital | Leaf | Leaf | canopy, or airflow through the canopy. |
| coordinates | | voucher | landmark | |
| Center horizontal | Digital | Leaf | Leaf | |
| landmark | | voucher | landmark | |
| coordinates | | | | |
| Area* | Digital | Leaf | Leaf shape | These are basic geometric morphometric |
| | | voucher | | shape traits. Traits with a * primarily are used |
| Convex hull area* | Digital | Leaf | Leaf shape | to determine the size and length/width of |
| | | voucher | | shape objects (currently leaves, but may be |
| Solidity | Digital | Leaf | Leaf shape | expanded to include branches or canopy). |
| | | voucher | | Traits with a * are primarily used to determine |
| Perimeter | Digital | Leaf | Leaf shape | how closely a shape resembles a circle vs how |
| | | voucher | | many holes or gaps exist within an ellipse, |
| Width* | Digital | Leaf | Leaf shape | which provides quantification for how densely |
| | | voucher | | lobed leaves are, if a leaf's leaflets are skinny |
| Height* | Digital | Leaf | Leaf shape | or thick, how airy or dense the canopy is, etc. |
| | | voucher | | ‘Estimated object count’ was designed to |
| Longest path* | Digital | Leaf | Leaf shape | count the number of leaflets for a given leaf, |
| | | voucher | | though it is currently not optimized for |
| Center of mass | Digital | Leaf | Leaf shape | Cannabis. Traits without demarcation are |
| | | voucher | | ways to determine if the program is collecting |
| Convex hull | Digital | Leaf | Leaf shape | data correctly. Size, shape, and density |
| vertices | | voucher | | characteristics are gateways to important |
| Ellipse center | Digital | Leaf | Leaf shape | agricultural traits - e.g., how large individual |
| | | voucher | | plants or cultivars are, how that affects |
| Ellipse major axis | Digital | Leaf | Leaf shape | spacing in the field, how much light can |
| length* | | voucher | | penetrate the canopy, or the amount of |
| Ellipse minor axis | Digital | Leaf | Leaf shape | biomass that is above the surface. Shape data |
| length* | | voucher | | can be used to inform relatedness to other |
| Ellipse major axis | Digital | Leaf | Leaf shape | cultivars and suggests there are implications to |
| angle* | | voucher | | light penetration and airflow through the |
| Ellipse eccentricity | Digital | Leaf | Leaf shape | canopy, though these require imaging of the |
| | | voucher | | whole canopy to make concrete observations. |
| Estimated object | Digital | Leaf | Leaf shape | |
| count | | voucher | | |
| Average number of | Manual | Leaf | Leaf shape | The number of leaflets per palmately-lobed |
| leaflets | | voucher, | | leaf can indicate plant vigor and health as well |
| | | branch | | as varies significantly between Cannabis |
| | | voucher | | cultivars. |
| Leaf serration | Manual | Leaf | Leaf shape | The morphology of leaf margins (edge of the |
| description | | voucher, | | leaf) varies significantly between Cannabis |
| | | branch | | cultivars and can be indicative of pedigree |
| | | voucher | | traits. |
| Average number of | Manual | Leaf | Leaf shape | |
| teeth of central | | voucher, | | |
| leaflet | | branch | | |
| | | voucher | | |
| Average central | Manual | Leaf | Leaf shape | Cannabis leaflets often vary between short |
| leaflet length | | voucher, | | and wide vs long and narrow. This |
| | | branch | | measurement standardizes a measurement of |
| | | voucher | | this trait, which often has a hereditary basis. |
| Average central | Manual | Leaf | Leaf shape | |
| leaflet width | | voucher, | | |
| | | branch | | |
| | | voucher | | |
| Description of | Manual | Live plant | Plant | The overall shape (including height and width) |
| canopy structure | | | architecture | of the canopy of as a whole varies widely |
| Average natural | Manual | Live plant | Plant | between Cannabis cultivars and is a desirable |
| height at maturity | | | architecture | trait to quantify impacts on yield, canopy |
| Average spread at | Manual | Live plant | Plant | management, and for best agricultural |
| maturity | | | architecture | practices. |
| Average number of | Manual | Inflorescence | Reproductive | Quantifying the size and floral compactness |
| flowers per | | video, | | (number of flowers) are useful traits for |
| inflorescence | | photos of | | determining yield. |
| | | live plant | | |
| Average length of | Manual | Live plant | Reproductive | |
| terminal | | | | |
| inflorescence | | | | |
| Average width of | Manual | Live plant | Reproductive | |
| terminal | | | | |
| inflorescence | | | | |
| Botanical scientific | Manual | Leaf | Leaf shape, | Blending aspects of all the traits listed above, |
| description | | voucher, | plant | this is a descriptive paragraph using scientific |
| | | branch | architecture, | terms designed to communicate life history |
| | | voucher | reproductive | and identification traits of the cultivar that can |
| | | | | be used for plant varietal patents. |

Collection method denotes if traits are collected digitally from an automated pipeline or collected manually by a specialist. Plant material measure describes if traits are collected from an herbarium voucher (either a voucher of leaves or a voucher of a branch), from a living plant, or from a photo or video of the live plant. Trait category is a general description of what type phenotype is collected.

TABLE 3
List of questions provided to requester for inclusion in the Cultivar Registration report.

| | Question Context (if | Type of | | |
|---|---|---|---|---|
| Question | applicable) | Question | Options if Multiple Choice | |
| Organization/Farm | | short answer | | |
| Growing Conditions | List all growing conditions | long answer | | |
| | utilized in the development | | | |
| | and growth of the cultivar, | | | |
| | such as indoor, outdoor, | | | |
| | etc. | | | |
| Light | List all light conditions | long answer | | |
| Conditions/Cycles | utilized in the cultivation of | | | |
| | the cultivar | | | |
| Soil Description | List all per growing | long answer | | |
| | condition | | | |
| Spacing | List the row spacing and | long answer | | |
| | plant spacing for each | | | |
| | growing condition | | | |
| Name of Cultivar | Cultivar that is the mother | short answer | | |
| Mother | of the cultivar | | | |
| Mother's Traits of | Mention any traits that were | long answer | | |
| Interest | desired in the cross to make | | | |
| | the cultivar | | | |
| Mother's Parents (if | Please let us know from | long answer | | |
| known) | which cultivars were bred | | | |
| | to yield the mother to help | | | |
| | context to the rich mosaic | | | |
| | of the cultivar's pedigree | | | |
| Origin of Mother | Please let us know from | short answer | | |
| (Farm/Organization, | where you acquired the | | | |
| if known) | mother to help context to | | | |
| | the rich mosaic of the | | | |
| | cultivar's pedigree | | | |
| Name of Cultivar | Cultivar that is the father of | short answer | | |
| Father | the cultivar | | | |
| Father's Traits of | Mention any traits that were | long answer | | |
| Interest | desired in the cross to make | | | |
| | the cultivar | | | |
| Father's Parents (if | Please let us know from | long answer | | |
| known) | which cultivars were bred | | | |
| | to yield the father to help | | | |
| | context to the rich mosaic | | | |
| | of the cultivar's pedigree | | | |
| Origin of Father | Please let us know from | short answer | | |
| (Farm/Organization, | where you acquired the | | | |
| if known) | father to help context to the | | | |
| | rich mosaic of the cultivar's | | | |
| | pedigree | | | |
| Breeding Setup | Briefly describe the | long answer | | |
| | breeding set-up used to | | | |
| | cross the parents | | | |
| Is this plant (or seed | | multiple | Yes | |
| set) your original | | choice | No | |
| breeding creation? | | | | |
| If not your original | | short answer | | |
| breeding creation, | | | | |
| please provide the | | | | |
| breeder's name. | | | | |
| How long have you | | long answer | | |
| stewarded this | | | | |
| plant/cultivar? | | | | |
| What do you love | | long answer | | |
| about this plant? | | | | |
| What is challenging | | long answer | | |
| about growing this | | | | |
| plant? | | | | |
| Are there similar | | long answer | | |
| cultivars? If so, how | | | | |
| does your plant | | | | |
| differ? | | | | |
| Propagation Method | This question refers to how | multiple | Seed | |
| | you intend for your cultivar | choice | Clone | |
| | to be grown and distributed | | | |
| Recommended | Rank: If you were to give | check box | Outdoor | Best/ideal |
| growth conditions | this plant to someone else | | Indoor | Acceptable |
| | to grow, which conditions | | Greenhouse | Poor |
| | would it do best in? | | Mixed Light | Unknown |
| Days to Germination | How many days did it take | short answer | | |
| | for your seed(s) to | | | |
| | germinate? If your plant is | | | |
| | intended for clonal | | | |
| | propagation, please provide | | | |
| | an estimate for the mother | | | |
| | plant's germination time, if | | | |
| | possible. | | | |
| Range of phenotypic | Rank how variable this | multiple | Low | |
| | plant is in light of the | choice | Medium | |
| | intended propagation | | High | |
| variability | method. If this is a seed | | | |
| | line, how uniform are the | | | |
| | seedlings? If this is a clone | | | |
| | line, do you notice variation | | | |
| | between cuttings? | | | |
| Proportion of | | multiple | Hermaphrodites | Low (5% or |
| hermaphrodite, | | choice | | less) |
| female, and male | | | Females | Low to |
| plants | | | | Medium (6- |
| | | | | 35%) |
| | | | Males | Medium (36- |
| | | | | 65%) |
| | | | | Medium to |
| | | | | High (66- |
| | | | | 95%″ |
| | | | | High (96% or |
| | | | | more) |
| | | | | Not |
| | | | | Applicable |
| Flowering time (days | Please include flowering | long answer | | |
| to flower after | times for all growing | | | |
| planting) | conditions that were | | | |
| | tested/utilized | | | |
| Productivity (average | Provide an estimate for all | long answer | | |
| or range lbs/plant) | of the growing conditions | | | |
| | utilized/tested | | | |
| Flower storage life | | short answer | | |
| Suitable processing | Select all that apply | multiple | Butane hash oil | Oil seed |
| categories for this | | choice | (BHO) | |
| plant | | | CBD extract | Resin |
| | | | Dry sift/dry | Rick Simpson |
| | | | sieve | oil (RSO) |
| | | | Fiber | Rosin |
| | | | Flower | Supercritical |
| | | | | CO2 oil |
| | | | Hash | Ticture |
| | | | Kief | Other . . . |
| Experience/User | Please include any flavors, | long answer | | |
| profile | feelings, and/or medicinal | | | |
| | qualities | | | |
| Olfactory | Select all that apply | multiple | Ammonia | Menthol |
| Characteristics | | choice | Apple | Mint |
| | | | Apricot | Nutty |
| | | | Berry | Orange |
| | | | Blue cheese | Peach |
| | | | Blueberry | Pear |
| | | | Butter | Pineapple |
| | | | Candy | Piney |
| | | | Cheese | Plum |
| | | | Chemical | Pomegranate |
| | | | Cherry | Pungent |
| | | | Chestnut | Rose |
| | | | Citrus | Sage |
| | | | Coffee | Skunk |
| | | | Diesel/gas | Spicy |
| | | | Earthy | Strawberry |
| | | | Floral | Sweet |
| | | | Fruity | Tar |
| | | | Grape | Tea |
| | | | Grapefruit | Tobacco |
| | | | Herbal | Tree Fruit |
| | | | Honey | Tropical Fruit |
| | | | Lavender | Vanilla |
| | | | Lemon | Violet |
| | | | Lime | Woody |
| | | | Mango | Other . . . |
| Average Stalk | Provide or estimate the | short answer | | |
| Diameter Size | thickness of the main stalk | | | |
| (inches) | if possible | | | |
| Stem Hollow (if | | multiple | Yes | |
| known) | | choice | No | |
| | | | Unknown | |
| Spongy Tissue inside | Pith is the spongy tissue | multiple | Absent | Thick |
| the Main Stalk when | that may be inside the main | choice | Thin | Unknown |
| Cut Open | stalk when it is cut open. If | | Medium | Other . . . |
| | known, select the option | | | |
| | that best describes the | | | |
| | consistency of the pith | | | |
| Depth of Stalk | | multiple | Shallow | None |
| Grooves | | choice | Medium | Other . . . |
| | | | Deep | |
| Stalk Color | | multiple | Yellow | Purple |
| | | choice | Medium Green | Other . . . |
| | | | Dark Green | |
| Stalk Trichome Type | Cystolithic hairs are slim | multiple | cystolithic hair | |
| or Hairiness | and curved, similar to a | choice | bulbous | |
| | bear claw. Bulbous | | capitate sessile | |
| | trichomes appear as a stalk | | capitate stalked | |
| | with a round top, similar to | | Other . . . | |
| | an ice cream cone. Capitate | | | |
| | sessile trichomes appear as | | | |
| | a ball shape directly on the | | | |
| | surface of the tissue. | | | |
| | Capitate stalked trichomes | | | |
| | are similar to bulbous | | | |
| | trichomes, but instead are | | | |
| | cinched at the junction of | | | |
| | the stalk and the ball shape | | | |
| Female Flower Color | | multiple | Purple | Green |
| | | choice | White | Other . . . |
| | | | Orange | |
| Additional Female | Use this space to describe | long answer | | |
| Flower Color | any unique variation that is | | | |
| Characteristics | observable | | | |
| Quantity of Female | Provide or estimate the | short answer | | |
| Flower per | average number of flowers | | | |
| Inflorescence | per bud | | | |
| Description of Cola | Color, hairiness/texture, | long answer | | |
| | glands, size estimate. | | | |
| | Include estimates for all | | | |
| | growing conditions | | | |
| | tested/utilized | | | |
| Male Bud Color at | | long answer | | |
| Maturity | | | | |
| Quantity of Male | An estimate of the average | short answer | | |
| Flowers per | number of flowers per | | | |
| Inflorescence | grouping of flowers (i.e., | | | |
| | inflorescence) | | | |
| Pollen Description | If applicable | long answer | | |
| Average Seed Size | | short answer | | |
| (mm, if known) | | | | |
| Average Weight per | | short answer | | |
| 1000 Seeds (g) | | | | |
| Seed Color | | multiple | Light Gray | Brown |
| | | choice | Medium Gray | Unknown |
| | | | Gray Brown | Other . . . |
| | | | Yellowish | |
| | | | Brown | |
| Seed Marbling | Weak means there are little | multiple | Weak | |
| | to no stripes or markings on | choice | Medium | |
| | the seeds. Medium means | | Strong | |
| | there are thin | | Unknown | |
| | stripes/markings on the | | Other . . . | |
| | seeds. Strong means there | | | |
| | are dark, thick | | | |
| | stripes/markings on the | | | |
| | seeds | | | |
| Seed | Add any unique details | long answer | | |
| Pattern/Morphology | about these seeds here (for | | | |
| Description | example, large | | | |
| | morphological variation, | | | |
| | texture, shape, if the | | | |
| | parianth, or old and | | | |
| | withered parts of the | | | |
| | flower, is persistent/still | | | |
| | clinging to the seeds) | | | |
| If possible, please | Please include a US coin in | attachment | | |
| include a photo of | the photo to provide a size | | | |
| seeds. | comparison | | | |
| Additional | | long answer | | |
| Phenotypes that Make | | | | |
| this Plant Unique (if | | | | |
| any) | | | | |
| Attach chemistry | If you have a standard plant | attachment | | |
| report | chemical report and wish to | | | |
| | share, please attach it. The | | | |
| | team recommends the | | | |
| | attachment of a chemical | | | |
| | report, as it increases the | | | |
| | validity of the botanical | | | |
| | description with the | | | |
| | addition of important, crop- | | | |
| | specific phenotypes and | | | |
| | will be included in your | | | |
| | final report | | | |
| At what stage in the | | short answer | | |
| growth cycle was the | | | | |
| plant sampled for | | | | |
| chemistry data? | | | | |
| Bacterial diseases | For all that apply, select if | multiple | Bacterial blight | Striatura |
| | your plant is “Very” or | | | ulcerosa |
| | “Somewhat” “Susceptible” | choice | Crown gall | Xanthomonas |
| | or “Resistant” | | | leaf spot |
| Fungal diseases | For all that apply, select if | multiple | Anthracnose | Phoma stem |
| | your plant is “Very” or | choice | | canker |
| | “Somewhat” “Susceptible” | | Black dot | Phomopsis |
| | or “Resistant” | | disease | stem canker |
| | | | Black mildew | Phymatotrich |
| | | | | um root rot |
| | | | Brown blight | Cotton root |
| | | | | rot |
| | | | Brown leaf spot | Pink rot |
| | | | & stem canker | |
| | | | Charcoal rot | Powdery |
| | | | | mildew |
| | | | Cladosporium | Red boot |
| | | | stem canker | |
| | | | Curvularia leaf | Rhizoctonia |
| | | | spot | soreshin & |
| | | | | root rot |
| | | | Cylindrosporium | Rust |
| | | | blight | |
| | | | Damping- | Southern |
| | | | off/Pythium rot | blight |
| | | | Downy mildew | Sclerotium |
| | | | | root & stem |
| | | | | rot |
| | | | Fusarium foot | Stemphylium |
| | | | rot & root rot | leaf & stem |
| | | | | rot |
| | | | Fusarium stem | Storage fungi |
| | | | canker | |
| | | | Fusarium wilt | Tar spot |
| | | | Gray mold (bud | Twig blight |
| | | | rot; Botrytis) | |
| | | | Hemp canker | Verticillium |
| | | | | wilt |
| | | | Leptosphaeria | White leaf |
| | | | blight | spot |
| | | | Olive leaf spot | Yellow leaf |
| | | | | spot |
| | | | Phiobolus stem | |
| | | | canker | |
| Phytoplasma diseases | For all that apply, select if | multiple | Witches' broom | |
| | your plant is “Very” or | choice | | |
| | “Somewhat” “Susceptible” | | | |
| | or “Resistant” | | | |
| Viral diseases | For all that apply, select if | multiple | Alfalfa mosaic | Cannabis |
| | your plant is “Very” or | choice | virus | cryptic virus |
| | “Somewhat” “Susceptible” | | Lettuce | Cucumber |
| | or “Resistant” | | chlorosis virus | mosaic virus |
| | | | Arabis mosaic | Hop latent |
| | | | virus | viroid |
| Nematodes | For all that apply, select if | multiple | Cyst | Root-knot |
| | your plant is “Very” or | choice | Needle | Stem |
| | “Somewhat” “Susceptible” | | | |
| | or “Resistant” | | | |
| Pests | For all that apply, select if | multiple | Aphids | Russet mites |
| | your plant is “Very” or | choice | Spider mites | Thread-foot or |
| | “Somewhat” “Susceptible” | | | white or |
| | or “Resistant” | | | tarsonemoid |
| | | | | mites |
| Add the susceptibility | | long answer | | |
| and resistance to any | | | | |
| other pests or | | | | |
| pathogens not listed | | | | |
| here | | | | |

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of identifying a Cannabis cultivar, comprising the steps of:
obtaining phenotypic data from one or more plants or plant parts from the cultivar; and/or
obtaining genotypic data from one or more plants or plant parts from the cultivar; and
assigning a cultivar designation based on the phenotypic data and/or the genotypic data,
thereby identifying the cultivar.

2. The method of claim 1, wherein the phenotypic data is in a digital form of the plant or a portion thereof.

3. The method of claim 1, wherein the phenotypic data comprises leaf size; plant size; flower; growth profile; fiber density, tensile strength, biofuel efficiency, phytoremediation use, nutritive potential, nutrient content, and/or ionomics.

4. The method of claim 1, wherein the genotypic data is obtained using polymerase chain reaction (PCR), next generation sequencing (NGS), restriction site associated DNA sequencing (RADseq), long read sequencing, nanopore long read sequencing, Sanger sequencing, restriction fragment length polymorphism (RFLP) analysis, oligonucleotide probes SNP chip array, microarray, and combinations thereof.

5. The method of claim 1, wherein the genotypic data comprises genetic analysis, transcriptional analysis, translational analysis, copy number variation analysis metabolomics analysis, proteomic analysis, epigenetic analysis, or combinations thereof.

6. The method of claim 1, further comprising determining genetic relationship information from the genotypic data.

7. The method of claim 1, further comprising transmitting the assigned cultivar designation to a requester or recipient.

8. The method of claim 7, wherein the requester or recipient is a grower, a government/regulatory agency, a dispensary, an individual, law enforcement, a researcher, a company, or a breeder.

9. The method of claim 1, further comprising providing, characterizing, confirming or denying breeding information.

10. The method of claim 1, further comprising providing, characterizing, confirming or denying ancestry information.

11. The method of claim 1, further comprising providing, characterizing, confirming or denying cultivar identity information.

12. The method of claim 1, further comprising providing, characterizing, confirming or denying supply chain information.

13. A method of identifying a Cannabis plant or portion thereof, comprising the steps of:
obtaining genotypic data from the plant or portion thereof; and
comparing the genotypic data obtained from the plant or portion thereof to reference genotypic data for Cannabis spp.,
thereby identifying the Cannabis plant or portion thereof.

14. The method of claim 13, wherein the genotypic data is obtained by sequencing genomic DNA from the plant or portion thereof.

15. The method of claim 13, further comprising validating or certifying the identity of the Cannabis plant or portion thereof.

16. The method of claim 13, further comprising determining if the Cannabis plant is clonal, a sibling, or a distant relative with respect to a reference plant or reference plant material.

17. A method of identifying a Cannabis plant, comprising the steps of:
obtaining genotypic data from the plant; and
comparing the genotypic data from the plant to one or more databases of genotypic data,
thereby identifying the Cannabis plant.

18. The method of claim 17, wherein the genotypic data is obtained by sequencing genomic DNA from the plant or portion thereof.

19. The method of claim 17, wherein the genotypic data is used to evaluate heterozygosity, genetic distance, and/or uniqueness.

20. The method of claim 17, wherein the identifying comprises identification of most likely cultivar, identification of most closely related cultivar with genetic similarities of certain features or attributes, identification of least closely related cultivar with genetic similarities of certain features or attributes.

21. A method of identifying or characterizing a Cannabis plant, comprising the steps of:
obtaining at least one image of the Cannabis plant;
determining a criteria for at least one phenotypic trait using the at least one image of the Cannabis plant; and
comparing the criteria for the at least one phenotypic trait of the Cannabis plant with at least one database of phenotypic traits,
thereby identifying or characterizing the Cannabis plant.

22. The method of claim 21, wherein the images are of whole plants.

23. The method of claim 21, wherein the images are obtained at a plurality of wavelengths.

24. The method of claim 21, wherein the phenotypic traits comprise leaf size; plant size; flower; or growth profile.

25. The method of claim 21, wherein the comparing is across a plurality of phenotypic traits.
